(19) United States
(12) Patent Application Publication
(10) Pub. No.: US 2004/0248839 A1
(43) Pub. Date: Dec. 9, 2004

(54) RNAi targeting of viruses

(75) Inventor: Timothy F. Kowalik, Princeton, MA (US)

Correspondence Address: LAHIVE & COCKFIELD, LLP., 28 STATE STREET, BOSTON, MA, 02109, US

(73) Assignee: UNIVERSITY OF MASSACHUSETTS, Worcester, MA (US)

(21) Appl. No.: 10/773,773

(22) Filed: Feb. 5, 2004

Related U.S. Application Data
(60) Provisional application No. 60/445,306, filed on Feb. 5, 2003.

(51) Int. Cl. A61K 48/00; C12N 15/86

(52) U.S. Cl. 514/44; 435/456

(57) ABSTRACT

The invention relates to methods and compositions that inhibit viral replication, e.g., CMV replication, within a host or host cell. Methods and compositions of the invention utilize RNA interference to block the translation of mRNA into proteins which are important or essential to viral replication. The method and compositions can be used to study CMV infection in in vitro cell culture and to treat CMV infection in non-human primates and human subjects.

### RELATED APPLICATIONS

This application claims the benefit of U.S. provisional patent application Ser. No. 60/445,306, entitled “RNAi Targeting of Viruses”, filed Feb. 5, 2003 (pending). The entire content of the above-referenced patent application is hereby incorporated by this reference.

### BACKGROUND OF THE INVENTION

Human cytomegalovirus (HCMV) is a member of the family of herpes viruses. HCMV is endemic within the human population and infection rarely causes symptomatic disease in immunocompetent individuals. However, HCMV infection in immunocompromised patients, including AIDS patients and transplant recipients, can have serious consequences. Infection in such patients can cause a variety of disorders, including pneumonitis, retinitis, disseminated viremia, and organ dysfunction. HCMV also poses a serious threat to the health of HIV-positive individuals because HCMV may accelerate the development of AIDS as well as contribute to the morbidity associated with increased. immunodeficiency. Likewise, HCMV infection can be problematic for pregnant women and children, especially infants. (Castillo and Kowalik, Gene 290:19-34 (2002))

The expression of HCMV genes occurs in a temporal order starting with immediate early (IE) genes, followed by the early genes, and finally, the late genes. The most abundant HCMV IE genes are from the unique long segment of the HCMV genome (UL). The IE transcripts arising from the UL123 region give rise to mRNA composed of four exons which encodes a 72 kDa nuclear phosphoprotein referred to as IE72. The IE transcripts arising from the UL122 region give rise to two major mRNA transcripts, one having the same first three exons as in the IE72 mRNA with exon 5 and encoding an 82-86 kDa nuclear protein, IE86, and the other encoding a 55 kDa protein, IE55, which is identical to IE86 except for a deletion resulting from a splicing event from exon 5. All three of the HCMV IE proteins (IE72, IE86, and IE55) share the same N-terminal 85 amino acid sequence, since they are encoded by the same first three exons. In general, HCMV IE genes are important for viral commitment to replication. IE72 and IE86 have been shown to be important for viral replication, while the function of IE55 is currently unknown.

### SUMMARY OF THE INVENTION

The compositions and methods described herein are based, in part, on the discovery that HCMV can be inhibited in an HCMV infected cell by the process of RNA interference (RNAi). The methods can be carried out by inhibiting viral (e.g, CMV, e.g., HCMV) proliferation (e.g, by inhibiting replication gene expression) with post-transcriptional inhibition such as RNA interference (RNAi). RNAi is induced by the introduction of siRNA (e.g., dsRNA or a vector expressing dsRNA) to infected cells. Most preferably, the dsRNA is of a length between about 18 and 29 nucleotides. In another aspect, the dsRNA has 5′ PO₄ and 3′ dTdT or 3′ TT.

The invention encompasses an isolated nucleic acid (e.g., a dsRNA or a vector or transgene expressing dsRNA) which includes or corresponds (e.g., complements) to the sequence of SEQ ID NO:1 and/or SEQ ID NO:2, or its complement.

The invention encompasses an RNAi agent, which induces RNAi within a cell, targeted to CMV nucleic acid. Targeted CMV nucleic acid molecules can include those expressing proteins that are important for viral survival, proliferation and replication (e.g., 1E1, 1E2, DNA polymerase, a scaffold protease, gB, and gH). The RNAi agent can be an siRNA (e.g, dsRNA, e.g., a dsRNA between about 18 and 29 nucleotides in length, or a vector expressing dsRNA, e.g., a plasmid DNA or viral vector expressing dsRNA between about 18 and 29 nucleotides in length). In another aspect, the siRNA is a dsRNA with 5′ PO₄ and 3′ dTdT or 3′ TT.

The invention encompasses methods of inhibiting expression of more than one gene simultaneously. In one aspect, RNAi targets an exon present in more than one mRNA transcript (e.g., exon 3, exon 2, or exon 1 of the genes UL122 and UL123 which encode IE72, IE86, and IE55). In another aspect, RNAi targets other genes important in viral survival, replication, and/or proliferation (e.g, 1E1, 1E2, DNA polymerase, a scaffold protease, gB, and gH).

The invention encompasses pharmaceutical compositions and methods of treating a CMV infected subject (e.g., a vertebrate mammal, a non-human primate or a human patient) by administering the pharmaceutical composition. The pharmaceutical compositions can include siRNA (e.g., dsRNA, e.g, a dsRNA between 18 and 29 nucleotides in length) or a vector expressing siRNA, and a pharmaceutically acceptable carrier. In another aspect, the siRNA is a dsRNA with 5′ PO₄ and 3′ dTdT or 3′ TT. The pharmaceutical compositions can be used to treat CMV associated conditions such as retinitis, pneumonitis, restenosis, cervical carcinoma, prostate cancer, adenocarcinoma of the colon, disseminated viremia, and organ dysfunction. In another aspect the pharmaceutical composition is administered in a localized or tissue-specific manner, such as intravitreal injection, to treat retinitis.

A gene or genes encoding “more than one protein” can include splice variants as well as proteins encoded by genes with different open reading frames that share a span of sequence such as an exon. As an example, UL122 and UL123 genes of CMV encode IE72, IE86, and IE55, each gene's open reading frame commonly using exon 3, exon 2, or exon 1.

Inhibition refers to decreased expression of a gene relative to endogenous levels or levels present in a CMV infected host, or complete block of gene expression. When using RNAi to inhibit a gene, it has been referred to in the art as gene expression “knock-down” and is used herein interchangeably with inhibition.

An RNAi agent is any agent than can induce RNA interference in a cell. Examples of RNAi agents are siRNA duplexes (e.g., dsRNA between about 18 and 29 nucleotides in length), shRNAs, miRNAs, ribozymes, antisense RNAs, etc.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is an illustration of the genetic structure of the UL122 and UL123 genes. As shown, Exon 1 through Exon 4 of UL 123 encode the IE72 protein; Exon 1, Exon 2, Exon 3, and Exon 5 of the UL 122 gene encode the IE86 protein; and Exon 2, Exon, 3, and a spliced Exon 5 encode the IE55 protein. IE72, IE86, and IE55 share the first N-terminal 85 amino acids.

FIG. 2A provides SEQ ID NO:1, which is the nucleic acid sequence for an RNAi agent (e.g, an siRNA or a duplex siRNA) that can be used to block expression of Exon 3 of UL122 or UL123. SEQ ID NO:1 is also referred to as “IEX3” or “X3” herein.

FIG. 2B provides SEQ ID NO:2, which is the nucleic acid sequence for an RNAi agent (e.g., an siRNA or a duplex siRNA) which can be used to block expression of Exon 3 of UL122 or UL123. SEQ ID NO:2 is also referred to as “IEY3” or “Y3” herein.

FIG. 3 is a Western blot of cell lysates from human fibroblasts infected with transgenic adenoviruses. The data show RNAi-mediated suppression of HCMV immediate early genes with siRNAs IEX3 (X3) and IEY3 (Y3), the sequences of which are provided in FIG. 2A and FIG. 2B, respectively. Diploid human fibroblasts, the model cell type for HCMV research, were electroporated with control or HCMV IE-specific siRNAs (X3 and Y3). These siRNAs target an exon shared by both UL122 and UL123 genes. Fibroblasts were infected 24 hours later with recombinant adenoviruses expressing UL123 (encoding IE1). At 48 hpi, cell lysates were generated and IE expression was examined by immunoblotting with an antibody specific for an epitope shared by IE1 and IE2. IE expression was greatly reduced in the presence of the X3 siRNA whereas only a modest reduction was seen upon treatment with Y3. The levels of expression in the “pum” lane exceed that achieved by high MOI infections with HCMV.

FIG. 4 is a bar graph showing the reduction of reporter gene expression by transfection of small interfering RNAs (siRNAs). COS cells were co-transfected with firefly luc and Renilla luc expressing plasmids along with siRNA to firefly luc or control siRNA (to Drosophila pumilio). Lysates were generated and luciferase activity determined. The x-axis shows the amount of siRNA transfected. Samples were normalized to Renilla luciferase activity and plotted as fold-inhibition relative to transfections with control siRNAs.

FIG. 5 is a Western blot showing RNAi suppression of HCMV IE gene expression during HCMV replication using the siRNAs IEX3, IEY3 or combinations of IEX3 and IEY3.

FIG. 6 is a Western blot showing that RNAi suppression of HCMV IE gene expression by the siRNA IEX3 results in suppression of glycoprotein B expression during HCMV infections.

FIG. 7 provides results of an experiment showing that RNAi suppression of HCMV IE gene expression results in reduced yields of progeny virus.

### DETAILED DESCRIPTION

As described herein, RNAi can be used to target an exon shared by multiple proteins, e.g., 2, 3, 4, or 5 proteins, with the expectation that expression of these multiple proteins can be simultaneously inhibited or aberrantly expressed, e.g., not expressed, expressed in a less than physiologically functional form, or expressed in a non-functional form, e.g., expressed in a non-functional truncated form. This can be generally applied to genome regions within infectious organisms, such as HCMV, from which multiple proteins are encoded.

The present invention is based on, but not limited to, the discovery that HCMV expression can be inhibited by targeting a single exon that is shared by multiple proteins required for HCMV commitment to replication within the host. By targeting this exon, e.g., by RNAi, more than one protein required for HCMV replication will be expressed aberrantly, e.g., not expressed, expressed in a less than physiological functional form, expressed in a non-functional form (e.g., expressed in a non-functional truncated form). Aberrant expression of proteins required for HCMV commitment to replication can impact the livelihood of the virus and reduce expression of HCMV within the host.

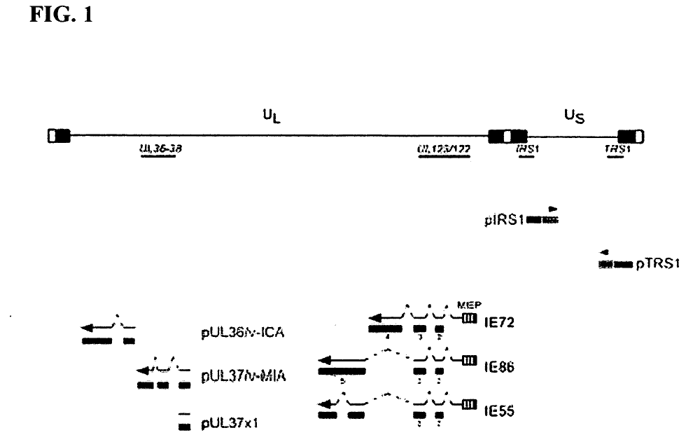

The HCMV immediate early proteins are required for the commitment to replication by the virus. The proteins having the greatest impact on HCMV replication are IE72 and IE86 encoded by the UL123 and UL122 open reading frames, respectively (see FIG. 1). Given the importance of these genes in virus replication, regions within a shared exon, for example Exon 3, can be selected as targets for inhibition of expression, for example, by RNAi response or antisense. Exon 3 encodes the N-terminal amino acids of IE72, IE86, and IE55. IE55 is poorly understood at this time.

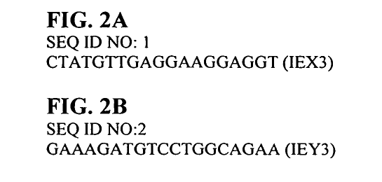

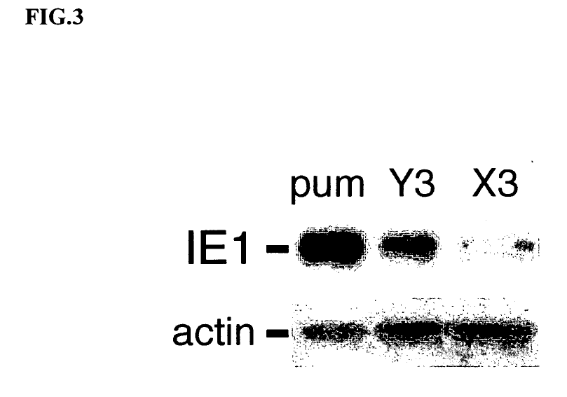

A stretch of sequence that is not likely to form stable secondary structures can be chosen for the generation of short interfering RNAs (siRNAs) for the purpose of inhibiting or decreasing expression of mRNA in more than one protein (e.g., Exon 3). Another guideline is that the GC content of the siRNA oligonucleotides be in the range of 30% and 70%. In one aspect, RNAi can be used to target specific sequences within Exon 3. RNAi can be used to target, for example, SEQ ID NO:1 (See, FIG. 2A, (IEX3)) and SEQ ID NO:2 (See FIG. 2B (IEY3)). The complement or RNA equivalent of SEQ ID NO:1 and SEQ ID NO:2 can also act as RNAi agents (e.g., siRNA duplexes). The inclusion of SEQ ID NO:1, SEQ ID NO:2, their complement, and/or their RNA Equivalent in larger molecules such as vector-based systems can also be used to generate an RNAi response. As described below, siRNAs can suppress or decrease the expression of the targeted open reading frames encoding IE proteins (termed X3 and Y3 in FIG. 3, respectively). Briefly, FIG. 3 is a western blot of IE1 protein encoded by UL123 and UL122 with actin as a control. The second lane shows inhibition by siRNA represented by the sequence of SEQ ID NO:2 and even greater inhibition in lane three by siRNA represented by the sequence of SEQ ID NO: 1.

CMV Genes and Targets

Human cytomegalovirus (HCMV) is a member of the Herpesviridae family of viruses. HCMV is an enveloped beta-herpesvirus with an approximately 230 kb double-stranded DNA genome containing approximately 200 open reading frames (ORFs). The HCMV genome is divided into two segments, designated UL (unique long) and US (unique short), bounded by inverted repeats.

Expression of HCMV genes occurs in a temporal order analogous to the other members of the Herpesviridae family. The first set of viral gene products to be expressed are classified as immediate early (IE) genes, followed by the expression of early (E) genes, and finally, the late (L) gene products. The IE genes do not require de novo protein synthesis for their expression. The most abundantly transcribed and best-characterized IE gene products originate from sequences encoded in the major IE region located within the UL segment of the viral genome (FIG. 1). Specifically, the major IE genes are encoded by ORFs that are under the control of the major IE promoter. Transcription from the major IE promoter through this region gives rise to several spliced mRNA species. The initial and most abundant transcript originates from the UL123 region and gives rise to a spliced 1.95 kb mRNA composed of exons 1 through 4 and encodes a 491 aa (72 kDa) nuclear phosphoprotein referred to as IE72 (also known, and referred to herein, as IE1 or IE1-72). Transcription through the other IE gene, UL122, gives rise to two major transcripts, a 2.25 and a 1.7 kb mRNA, that have the same first three exons as in the IE72 mRNA but contain a novel exon, exon 5, in place of exon 4 as a result of alternative splicing. The 2.25 kb mRNA encodes a 579 aa (82-86 kDa) nuclear protein, IE86 (also known, and referred to herein, as IE2 or IE2-86), and the 1.7 kb mRNA encodes for a 425 aa (55 kDa) protein, IE 55 (also known, and referred to herein, as IE2-55). IE55 is identical to IE86 except for a 154 aa deletion between residues aa 365 and 519 resulting from a splicing event within exon 5. Transcription from a cryptic start site within exon 5 generates a transcript that encodes for a 338 aa (40 kDa) protein that is expressed as a late gene product. Because all three of the HCMV IE proteins contain the same first three exons, they all share the same 85 aa in their N-terminal sequence. However, the remaining sequences in each of the IE proteins differ and likely account for the divergent activities exhibited by each protein.

CMV early (E) genes include UL54, encoding a DNA polymerase, and UL97, encoding a protein that phosphorylates ganciclovir. CMV(L) late genes include UL80, encoding a protease, UL55, encoding the attachment protein glycoprotein B (gB), and UL75, encoding the attachment protein glycoprotein H (gH).

CMV Targets

The nucleic acid targets of siRNAs as described herein may be any gene of a Herpesviridae, e.g., any gene of Betaherpesvirinae, e.g., any gene of Human herpesvirus 5 or Human cytomegalovirus (HCMV). In a preferred embodiment of the invention, the nucleic acid targets are HCMV genes. HCMV genes which are targets of siRNAs of the invention can be genes of any HCMV strain, for example, HCMV strains including, but not limited to, HCMV AD 169 strain, Towne strain, Toledo strain, and Merlin strain.

In one embodiment, the siRNA of the invention inhibits the synthesis of viral CMV (e.g., HCMV) RNA transcripts. In another, the siRNA promotes the degradation of or inhibits synthesis of viral CMV (e.g., HCMV) RNA transcripts. In yet another, the siRNA blocks the translation of viral CMV (e.g., HCMV) RNA transcripts. The siRNA can mediate RNAi during an early viral replication cycle event and/or a late viral replication cycle event.

The target portion of the CMV genome can be the portion of the genomic DNA that specifies the amino acid sequence of a viral CMV protein or enzyme (e.g., encoding one or more of the group consisting of IE1, 1E2, DNA polymerase, a scaffold protease, glycoprotein B, and glycoprotein H). As used herein, the phrase “specifies the amino acid sequence” of a protein means that the RNA sequence is translated into the amino acid sequence according to the rules of the genetic code. The protein may be a viral protein involved in immunosuppression of the host, replication of CMV, transmission of the CMV, or maintenance of the infection.

Preferably, the target portion of the CMV genome is a highly conserved region. Also within the scope of the invention, CMV virus can be extracted from a patient and the siRNA can be produced to match a portion of the CMV genome that has mutated. This can be done for generations of CMV mutations to mediate RNAi in a patient that develops resistance to previously used siRNAs. It is also within the scope of the invention that series of siRNAs are introduced to a cell or organism. When a series of siRNAs are used, preferably the series of siRNAs correspond to one or more highly conserved region of the CMV genome. When targeting highly conserved regions, relatively few siRNAs can be effective in mediating RNAi despite mutations in the genome.

Examples of HCMV genes that may be targets of siRNAs of the invention include, but are not limited to, TRL1 RL1, TRL2 RL2, TRL3 RL3, TRL5 RL5, RL5A, TRL4 RL4, TRL6 RL6, RL7 TRL7, TRL8, TRL9 RL9, RL10 TRL10, RL11 TRL11, TRL12 RL12, TRL13 RL13, TRL14 RL14, UL1, UL2, UL3, UL4, UL5, UL6, UL 7, UL8, UL9, UL10, UL11, UL13, UL12, UL14, UL16, UL15A, UL17, UL18, UL19, UL20, UL21A UL21.5, UL22A, UL23, UL24, UL25, UL26, UL27, UL28, UL29, UL30, UL31, UL32, UL33, UL34, UL35, UL35A, UL36, UL38, UL37 gpUL37, UL39, UL40, UL41A, UL42, UL43, UL44, UL45, UL46, UL47, UL48, UL48.5 UL48/UL49 UL48A, UL49, UL50, UL51, UL52, UL53, UL54, UL55 gB, UL56, UL57 ICP8 ssDNA BP, UL58, UL59, UL60, UL61, UL62, UL63, UL64, UL65, UL66, UL67, UL68, UL69, UL71, UL70, UL72, UL73 gN, UL74 gO, UL75 gH, UL76, UL77, UL78, UL79, UL80 apnG, UL80.5 UL80a, UL81, UL82, UL83, UL84, UL85, UL86, UL87, UL88, UL91, UL90, UL92, UL93, UL94, UL95, UL89, UL96, UL97, UL98, UL99, gM UL100, UL102, UL103, UL105, UL104, UL106, UL107, UL108, UL109, UL110, UL111A cmvIL-10, UL111, UL112, UL114 UDG, UL115, UL116, UL117, UL 119, UL120, UL121, UL122, IE1 UL123, UL124, UL125, UL126, UL127, UL128, UL129, UL130, UL131A, UL132, UL148, RL13 IRL13, RL12 IRL12, RL11 IRL11, IRL10 RL10, IRL9 RL9, IRL8 RL8, IRL7 RL7, RL6 IRL6, IRL4 RL4, RL5 IRL5, IRL3 RL3, IRL2, RL1 IRL1, J1I, IRS1, US1, US2, US3, US4, US5, US6, US7, US8, US9, US10, US11, US12, US13, US14, US15, US16, US17, US18, US19, US20, US21, US22, US23, US24, US25, US26, US27, US28, US29, US30, US31, US32, US34, US34A, US33, US35, US36, TRS1, J1S, UL133, UL135, UL134, UL136, UL138, UL137, UL139, UL140, UL141, UL142, UL143, UL144 ppUL144, UL145, vCXC-1 UL146, UL147, UL147A, UL148, UL132, UL130, UL149, UL150, UL151, UL147A, UL147, UL152, UL153, and UL154.

In various embodiments, HCMV genes which are targets of siRNAs of the invention include, but are not limited to, e.g., UL123, UL122, UL54, UL97, UL80, UL55 and UL75. In various embodiments, examples of HCMV genes include, but are not limited to, e.g., a gene encoding 1E1, 1E2, DNA polymerase, ppUL97, a scaffold protease, glycoprotein B (gB), and glycoprotein H (gH). In particular embodiments, the target genes comprise the target nucleotide sequences shown in Table 1.

In various embodiments, target portions of the CMV (e.g., HCMV) genome include, but are not limited to, the UL122 and UL123 genes of CMV, which encode the proteins IE72, IE86, and IE55, wherein each gene's open reading frame commonly uses exon 1, exon 2, or exon 3. In a preferred embodiment, the target portion of the CMV genome is a region (e.g., exon) which is present in an mRNA, wherein the mRNA is translated into more than one protein (e.g., exon 1, exon 2 or exon 3 of the UL122 and UL123 genes, wherein UL122 and UL123 encode the EI72, IE86 and IE55 proteins). In this way, at least two or more proteins can be inhibited by a single RNAi agent.

Accordingly, the DNA sequence of UL123 can be, for example, the sequences substantially identical to HCMV AD169 strain UL123, including but not limited to GenBank Accession No. NC_{—}001347, GeneID: 1487822 (SEQ ID NO:177). The DNA sequence of UL122 can be, for example, the sequences substantially identical to HCMV AD169 strain UL122, including but not limited to GenBank Accession No. NC_{—}001347, GeneID: 1487821 (SEQ ID NO:178). The DNA sequence of UL54 can be, for example, the sequences substantially identical to HCMV AD169 strain UL54, including but not limited to GenBank Accession No. NC_{—}001347, GeneID:1487749 (SEQ ID NO:179). DNA sequence of UL97 can be, for example, the sequences substantially identical to HCMV AD169 strain UL97, including but not limited to GenBank Accession No. NC_{—}001347, GeneID: 1487738 (SEQ ID NO:180). The DNA sequence of UL80 can be, for example, the sequences substantially identical to HCMV AD169 strain UL80, including but not limited to GenBank Accession No. NC_{—}001347, GeneID: 1487752 (SEQ ID NO:181). The DNA sequence of UL55 can be, for example, the sequences substantially identical to HCMV AD169 strain UL55, including but not limited to GenBank Accession No. NC_{—}001347, GeneID: 1487750 (SEQ ID NO:182). The DNA sequence of UL75 can be, for example, the sequences substantially identical to HCMV AD169 strain UL75, including but not limited to GenBank Accession No. NC_{—}001347, GeneID: 1487831 (SEQ ID NO:183).

In exemplary embodiments, the siRNA molecules of the present invention can target the following sequences of the target gene, e.g., the siRNA molecules may comprise, as one of their strands, an RNA sequence corresponding to any one of the following DNA sequences (e.g., the sense strand of the siRNA duplex) and the corresponding sequences of allelic variants thereof. Sequences in the table are represented as target gene sequences (i.e., DNA sequences). The skilled artisan will appreciate, however, that siRNA strands, e.g., sense strands, comprise corresponding ribonucleotides, and that antisense strands comprise complementary ribonucleotide sequences. Additional deoxythymidine overhangs, e.g., 3′ dTdT overhangs, are also contemplated as described herein.

TABLE I
siRNA CANDIDATE TARGET SEQUENCES in HCMV

| | siRNA CANDIDATE | |
|---|---|---|
| GENES | TARGET SEQUENCES | SEQUENCES |
| UL123 | GAACTCGTCAAACAGATTA | SEQ ID NO:7 |
| | ACTCGTCAAACAGATTAAG | SEQ ID NO:8 |
| | CTCGTCAAACAGATTAAGG | SEQ ID NO:9 |
| | TGGTGCGGCATAGAATCAA | SEQ ID NO:10 |
| | GACGGAAGAGAAATTCACT | SEQ ID NO:11 |
| | GAAATTCACTGGCGCCTTT | SEQ ID NO:12 |
| | AATTCACTGGCGCCTTTAA | SEQ ID NO:13 |
| | ATTCACTGGCGCCTTTAAT | SEQ ID NO:14 |
| | TTCACTGGCGCCTTTAATA | SEQ ID NO:15 |
| | GCCTTTCGAGGAGATGAAG | SEQ ID NO:16 |
| | CATTGTACCTGAGGATAAG | SEQ ID NO:17 |
| | TTAAGGAGCTGCATGATGT | SEQ ID NO:18 |
| | AGGATGAACTTAGGAGAAA | SEQ ID NO:19 |
| | ACTTAGGAGAAAGATGATG | SEQ ID NO:20 |
| | CTTAGGAGAAAGATGATGT | SEQ ID NO:21 |
| | TTTATGGATATCCTCACTA | SEQ ID NO:22 |
| | AACAATGTGTAATGAGTAC | SEQ ID NO:23 |
| | ATGAGTACAAGGTCACTAG | SEQ ID NO:24 |
| | TGAGTACAAGGTCACTAGT | SEQ ID NO:25 |
| | GTGACGCTTGTATGATGAC | SEQ ID NO:26 |
| | AGCGGCCTCTGATAACCAA | SEQ ID NO:27 |
| | ACCAAGCCTGAGGTTATCA | SEQ ID NO:28 |
| UL122 | ATCATGCCGGTATCGATTC | SEQ ID NO:29 |
| | AAACCACGCGTCCTTTCAA | SEQ ID NO:30 |
| | AACCACGCGTCCTTTCAAG | SEQ ID NO:31 |
| | CCATCCAGTACCGCAACAA | SEQ ID NO:32 |
| | GTACCGCAACAAGATTATC | SEQ ID NO:33 |
| | CCGCAACAAGATTATCGAT | SEQ ID NO:34 |
| | AGAAGAGCAAACGCATCTC | SEQ ID NO:35 |
| | AACGCATCTCCGAGTTGGA | SEQ ID NO:36 |
| | CAACGAGAAGGTGCGCAAT | SEQ ID NO:37 |
| | CACCAATCGCTCTCTTGAG | SEQ ID NO:38 |
| | CCAATCGCTCTCTTGAGTA | SEQ ID NO:39 |
| | ATCGCTCTCTTGAGTACAA | SEQ ID NO:40 |
| | CCATGCAGGTGAACAACAA | SEQ ID NO:41 |
| | CAGCCGATGCTTGTAACGA | SEQ ID NO:42 |
| | TTACCGCAACATGATCATC | SEQ ID NO:43 |
| UL123/UL122 | CTATGTTGAGGAAGGAGGT | SEQ ID NO:1 |
| (exons 1, | GAAAGATGTCCTGGCAGAA | SEQ ID NO:2 |
| 2 or 3) | CGACGTTCCTGCAGACTAT | SEQ ID NO:3 |
| | TGTTGAGGAAGGAGGTTAA | SEQ ID NO:4 |
| | GGAAGGAGGTTAACAGTCA | SEQ ID NO:5 |
| | CAAGTGACCGAGGATTGCA | SEQ ID NO:6 |
| UL54 | TGTTCTATCGAGAGATTAA | SEQ ID NO:44 |
| | CAGAACACGGCTACAGTAT | SEQ ID NO:45 |
| | GAACACGGCTACAGTATCT | SEQ ID NO:46 |
| | CTTGTGATATCGAGGTAGA | SEQ ID NO:47 |
| | TCGAGGTAGACTGCGATGT | SEQ ID NO:48 |
| | TGCCTGTCCTTCGATATCG | SEQ ID NO:49 |
| | ACACTATGGCCGAGCTTTA | SEQ ID NO:50 |
| | CACTATGGCCGAGCTTTAC | SEQ ID NO:51 |
| | TTGGTGCGCGATCTGTTCA | SEQ ID NO:52 |
| | ACGAATAGCGTTGCTGTGT | SEQ ID NO:53 |
| | CCTAACGCTGCTATCATCT | SEQ ID NO:54 |
| | ATGCATGCGCGAGTGTCAA | SEQ ID NO:55 |
| | ACAGATGGCGCTCAAAGTA | SEQ ID NO:56 |
| | AAGTAACGTGCAACGCTTT | SEQ ID NO:57 |
| | AGTAACGTGCAACGCTTTC | SEQ ID NO:58 |
| | GTAACGTGCAACGCTTTCT | SEQ ID NO:59 |
| | AAAGGTCTTCGTCTCTCTT | SEQ ID NO:60 |
| | AAGGTCTTCGTCTCTCTTA | SEQ ID NO:61 |
| | TGATCTGCAAGAAACGTTA | SEQ ID NO:62 |
| | TCTGCAAGAAACGTTACAT | SEQ ID NO:63 |
| | AACGTTACATCGGCAAAGT | SEQ ID NO:64 |
| | ACGTTACATCGGCAAAGTG | SEQ ID NO:65 |
| | CATCTCGCTGTACCGTCAA | SEQ ID NO:66 |
| | TCTCGCTGTACCGTCAATC | SEQ ID NO:67 |
| | TTGCCGTCATTAAGCGATT | SEQ ID NO:68 |
| | CGCCGACAAGTACTTTGAG | SEQ ID NO:69 |
| UL97 | TTTGTTATGCCGTGGACAT | SEQ ID NO:70 |
| | CAACGTCACGGTACATCGA | SEQ ID NO:71 |
| | CGGTACATCGACGTTTCCA | SEQ ID NO:72 |
| | ATCACCAGTGTCGTGTATG | SEQ ID NO:73 |
| | TCACCAGTGTCGTGTATGC | SEQ ID NO:74 |
| | GTGTCGTGTATGCCACTTT | SEQ ID NO:75 |
| | TGCCACTTTGACATTACAC | SEQ ID NO:76 |
| | CGGAGGCGTTGCTCTTTAA | SEQ ID NO:77 |
| UL80 | AAAGTCCGAGCTGGTTTCG | SEQ ID NO:78 |
| | TACGTCAAGGCGAGCGTTT | SEQ ID NO:79 |
| | ACAAACGCCGTAAGGAAAC | SEQ ID NO:80 |
| | CAAACGCCGTAAGGAAACC | SEQ ID NO:81 |
| | GCAGCAGCAACAACGTTAC | SEQ ID NO:82 |
| | GCAACAACGTTACGATGAA | SEQ ID NO:83 |
| | GAGTTCTACGTTACTTTCG | SEQ ID NO:84 |
| | CTACTACTACCGTGTGTAC | SEQ ID NO:85 |
| | GACATGGTAGATCTGAATC | SEQ ID NO:86 |
| UL55 | GTCTGCGTTAACCTGTGTA | SEQ ID NO:87 |
| | AGCCATACTTCTCGTACGA | SEQ ID NO:88 |
| | TAGAGCCAACGAGACTATC | SEQ ID NO:89 |
| | GAGCCAACGAGACTATCTA | SEQ ID NO:90 |
| | GCCAACGAGACTATCTACA | SEQ ID NO:91 |
| | ACGAGACTATCTACAACAC | SEQ ID NO:92 |
| | CGAGACTATCTACAACACT | SEQ ID NO:93 |
| | CGGATCTTATTCGCTTTGA | SEQ ID NO:94 |
| | TCTTATTCGCTTTGAACGT | SEQ ID NO:95 |
| | TTCGCTTTGAACGTAATAT | SEQ ID NO:96 |
| | CCTCGATGAAGCCTATCAA | SEQ ID NO:97 |
| | TGAAGCCTATCAATGAAGA | SEQ ID NO:98 |
| | TCAACAAGTTTGCTCAATG | SEQ ID NO:99 |
| | GTTCCTACAGCCGCGTTAT | SEQ ID NO:100 |
| | TCGTGAGACCTGTAATCTG | SEQ ID NO:101 |
| | ACTGTATGCTGACCATCAC | SEQ ID NO:102 |
| | CTGTATGCTGACCATCACT | SEQ ID NO:103 |
| | ACGGAACCAATCGCAATGC | SEQ ID NO:104 |
| | AGCCTCGGAACGTACTATC | SEQ ID NO:105 |
| | CGTGATGAGGCTATAAATA | SEQ ID NO:106 |
| | AACGTGTCCGTCTTCGAAA | SEQ ID NO:107 |
| | ACGTGTCCGTCTTCGAAAC | SEQ ID NO:108 |
| | CGTTTGGCCAATCGATCCA | SEQ ID NO:109 |
| | ATCGATCCAGTCTGAATAT | SEQ ID NO:110 |
| | TCGATCCAGTCTGAATATC | SEQ ID NO:111 |
| | GAAGTACGAGTGACAATAA | SEQ ID NO:112 |
| | GTACGAGTGACAATAATAC | SEQ ID NO:113 |
| | GCATGGAATCGGTGCACAA | SEQ ID NO:114 |
| | TGGAATCGGTGCACAATCT | SEQ ID NO:115 |
| | CGTTGCGCGGTTACATCAA | SEQ ID NO:116 |
| | TTTACAACAAACCGATTGC | SEQ ID NO:117 |
| | GGTGCTGCGTGATATGAAC | SEQ ID NO:118 |
| | ATTTCGCCAACAGCTCGTA | SEQ ID NO:119 |
| | ACAGCTCGTACGTGCAGTA | SEQ ID NO:120 |
| | GTACGTGGACTACCTCTTC | SEQ ID NO:121 |
| | CGTGGACTACCTCTTCAAA | SEQ ID NO:122 |
| | AGAGATCATGCGCGAATTC | SEQ ID NO:123 |
| | GAGATCATGCGCGAATTCA | SEQ ID NO:124 |
| | GATCATGCGCGAATTCAAC | SEQ ID NO:125 |
| | TCATGCGCGAATTCAACTC | SEQ ID NO:126 |
| | TGCGCGAATTCAACTCGTA | SEQ ID NO:127 |
| | AGTACGTGGAGGACAAGGT | SEQ ID NO:128 |
| | GTACGTGGAGGACAAGGTA | SEQ ID NO:129 |
| | TAGCCGTAGTCATTATCAC | SEQ ID NO:130 |
| | GCCGTAGTCATTATCACTT | SEQ ID NO:131 |
| | CCAAAGACACGTCGTTACA | SEQ ID NO:132 |
| | GAACGGTACAGATTCTTTG | SEQ ID NO:133 |
| | AACGGCTACAGACACTTGA | SEQ ID NO:134 |
| | CTTGAAAGACTCCGACGAA | SEQ ID NO:135 |
| | CTCCGACGAAGAAGAGAAC | SEQ ID NO:136 |
| UL75 | CCTACCTTCGCAACGATAT | SEQ ID NO:137 |
| | CGCATTTCACCTACTACTC | SEQ ID NO:138 |
| | TTCCATATGCCTCGATGTC | SEQ ID NO:139 |
| | GGTAGATCTGACCGAAACC | SEQ ID NO:140 |
| | CTTAACACCTACGCATTGG | SEQ ID NO:141 |
| | ACACCTACGCATTGGTATC | SEQ ID NO:142 |
| | CTACATCGGCCACACTTTA | SEQ ID NO:143 |
| | CCTCATGGACGAACTACGT | SEQ ID NO:144 |
| | TCAACGCGACAACTTTATA | SEQ ID NO:145 |
| | CAACTTTATACTACGACAA | SEQ ID NO:146 |
| | ACTTTATACTACGACAAAC | SEQ ID NO:147 |
| | GCTCCTGGTACTAGTTAAG | SEQ ID NO:148 |
| | CTAGTTAAGAAAGCTCAAC | SEQ ID NO:149 |
| | GCTCAACTAAACCGTCACT | SEQ ID NO:150 |
| | AACCGTCACTCCTATCTCA | SEQ ID NO:151 |
| | CCGTCACTCCTATCTCAAA | SEQ ID NO:152 |
| | CGCTGTAGACGTACTCAAA | SEQ ID NO:153 |
| | AGCGGTCGATGTCAAATGT | SEQ ID NO:154 |
| | GCGGTCGATGTCAAATGTT | SEQ ID NO:155 |
| | GGCCGCACTCTTACAAATA | SEQ ID NO:156 |
| | TGATCACCTGCCTCTCACA | SEQ ID NO:157 |
| | GAGACGCGAAATCTTCATC | SEQ ID NO:158 |
| | GACGCGAAATCTTCATCGT | SEQ ID NO:159 |
| | TTGGCCGAGCTATCACACT | SEQ ID NO:160 |
| | CTTTACGCAGTTGCTAGCT | SEQ ID NO:161 |
| | ATACCTCAGCGACCTGTAC | SEQ ID NO:162 |
| | TACCTCAGCGACCTGTACA | SEQ ID NO:163 |
| | ACACGTCAGTTATGTCGTA | SEQ ID NO:164 |
| | CACGTCAGTTATGTCGTAA | SEQ ID NO:165 |
| | AACGGACAGTCAAACTAAA | SEQ ID NO:166 |
| | ACGGACAGTCAAACTAAAT | SEQ ID NO:167 |
| | CGGACAGTCAAACTAAATG | SEQ ID NO:168 |
| | CGCAAGGCGTCATCAACAT | SEQ ID NO:169 |
| | CAACGAAGTGGTGGTCTCA | SEQ ID NO:170 |
| | AAACGGTACGGTCCTAGAA | SEQ ID NO:171 |
| | AACGGTACGGTCCTAGAAG | SEQ ID NO:172 |
| | ACGGTACGGTCCTAGAAGT | SEQ ID NO:173 |
| | CGGTACGGTCCTAGAAGTA | SEQ ID NO:174 |
| | CAGTCGTCTCCTCATGATG | SEQ ID NO:175 |
| | GTCGTCTCCTCATGATGTC | SEQ ID NO:176 |

RNA Interference

RNAi is a remarkably efficient process whereby double-stranded RNA (dsRNA) induces the sequence-specific degradation of homologous mRNA in animals and plant cells (Hutvagner and Zamore, Curr. Opin. Genet. Dev., 12:225-232, 2002; Sharp, Genes Dev. 15:485-490,2001). In mammalian cells, RNAi can be triggered by 21-nucleotide (nt) duplexes of small interfering RNA (siRNA) (Chiu et al, Mol Cell 10:549-561, 2002; Elbashir et al., Nature 411:494-498, 2001), or by micro-RNAs (miRNA), functional small-hairpin RNA (shRNA), or other dsRNAs that are expressed in vivo using DNA templates with RNA polymerase III promoters (Zeng et al., Mol. Cell 9:1327-1333, 2002; Paddison et al., Genes Dev. 16:948-958, 2002; Lee et al., Nature Biotechnol. 20:500-505, 2002; Paul et al., Nature Biotechnol. 20:505-508, 2002; Tuschl, Nature Biotechnol. 20:440-448, 2002; Yu et al. Proc.Natl. Acad. Sci. USA 99:6047-6052, 2002; McManus et al., RNA 8:842-850, 2002; Sui et al., Proc. Natl. Acad Sci. USA 99:5515-5520, 2002).

Suppliers of RNA synthesis reagents and synthesized RNA oligonucleotides include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK).

Nucleic Acid Molecules

1. siRNA Molecules.

The present invention features siRNA molecules, methods of making siRNA molecules and methods (e.g., research and/or therapeutic methods) for using siRNA molecules. The siRNA molecule can have a length from about 10-50 or more nucleotides (or nucleotide analogs), about 16-30 nucleotides (or nucleotide analogs), about 15-25 nucleotides (or nucleotide analogs), or about 20-23 nucleotides (or nucleotide analogs). The nucleic acid molecules or constructs of the invention include dsRNA molecules that have nucleotide (or nucleotide analog) lengths of about 10-20, 20-30, 30-40, 40-50, 15, 16, 17, 18, 19, 20, 21, 22, 23, 24, 25, 26, 27, 28, 29, 30 or more. In a preferred embodiment, the siRNA molecule has a length of 21 nucleotides. It is to be understood that all ranges and values encompassed in the above ranges are within the scope of the present invention. Long dsRNAs to date generally are less preferable as they have been found to induce cell self-destruction known as interferon response in human cells. siRNAs can preferably include 5′ terminal phosphate (e.g., 5′ PO₄) and a 3′ short overhangs of about 2 nucleotides (e.g., 3′-deoxythymidines, e.g., 3′ dTdT overhangs). The dsRNA molecules of the invention can be chemically synthesized, transcribed in vitro from a DNA template, or made in vivo from, for example, shRNA. In a preferred embodiment, the siRNA can be a short hairpin siRNA (shRNA). Even more preferably, the shRNA is an expressed shRNA. In another embodiment, the siRNA can be associated with one or more proteins in an siRNA complex. In an exemplary embodiment, the siRNA target region is Exon 3 of the HCMV UL122 and UL123 genes.

The siRNA molecules of the invention include a sequence that is sequence sufficiently complementary to a portion of the viral (e.g., CMV, e.g., HCMV) genome to mediate RNA interference (RNAi), as defined herein, i.e., the siRNA has a sequence sufficiently specific to trigger the degradation of the target RNA by the RNAi machinery or process. The siRNA molecule can be designed such that every residue of the antisense strand is complementary to a residue in the target molecule. Alternatively, substitutions can be made within the molecule to increase stability and/or enhance processing activity of said molecule. Substitutions can be made within the strand or can be made to residues at the ends of the strand.

The target RNA cleavage reaction guided by siRNAs is highly sequence specific. In general, siRNAs containing nucleotide sequences substantially complementary to a portion of the target gene, e.g., target region of an HCMV mRNA, are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition as shown in the examples. Alternatively, siRNA sequences with nucleotide analog substitutions or insertions can be effective for inhibition. For example the first and second strands can be about 80% (e.g., 85%, 90%, 95%, or 100%) complementary to a target region of HCMV mRNA (e.g., the sequence of a strand of the dsRNA and the sequence of the target can differ by 0, 1, 2, or 3 nucleotide(s)).

Moreover, not all positions of a siRNA contribute equally to target recognition. Mismatches in the center of the siRNA are most critical and can essentially abolish target RNA cleavage. In contrast, the 3′ nucleotides of the siRNA typically do not contribute significantly to specificity of the target recognition. In particular, 3′ residues of the siRNA sequence which are complementary to the target RNA (e.g., the guide sequence) generally are not critical for target RNA cleavage.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin & Altschul, Proc. Natl. Acad Sci. USA 87:2264-68 (1990), modified as in Karlin & Altschul, Proc. Natl. Acad. Sci. USA 90:5873-77 (1993). Such an algorithm is incorporated into the BLAST programs (version 2.0) of Altschul, et al., J. Mol. Biol. 215:403-10 (1990).

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul, et al., Nucleic Acids Res. 25(17):3389-3402 (1997). In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Greater than 90% sequence identity, e.g., 91%, 92%, 93%, 94%, 95%, 96%, 97%, 98%, 99% or even 100% sequence identity, between the siRNA and the portion of the target gene is preferred. For example, in the context of an siRNA of about 19-25 nucleotides, e.g., at least 15-21 identical nucleotides are preferred, more preferably at least 17-22 identical nucleotides, and even more preferably at least 18-23 or 19-24 identical nucleotides. Alternatively worded, in an siRNA of about 19-25 nucleotides in length, siRNAs having no greater than about 5 mismatches are preferred, preferably no greater than 4 mismatches are preferred, preferably no greater than 3 mismatches, more preferably no greater than 2 mismatches, and even more preferably no greater than 1 mismatch.

Alternatively, the siRNA may be defined functionally as a nucleotide sequence (or oligonucleotide sequence) that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log10[Na+])+0.41(% G+C)-(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., et al., 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel, et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 or 50 bases.

In one embodiment, the RNA molecules of the present invention are modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference. For example, the absence of a 2′ hydroxyl may significantly enhance the nuclease resistance of the siRNAs in tissue culture medium.

In an especially preferred embodiment of the present invention the RNA molecule may contain at least one modified nucleotide analogue. The nucleotide analogues may be located at positions where the target-specific activity, e.g., the RNAi mediating activity is not substantially effected, e.g., in a region at the 5′-end and/or the 3′-end of the RNA molecule. Particularly, the ends may be stabilized by incorporating modified nucleotide analogues.

Preferred nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

Also preferred are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O— and N-alkylated nucleotides, e.g., N6-methyl adenosine are suitable. It should be noted that the above modifications may be combined.

Crosslinking can be employed to alter the pharmacokinetics of the composition, for example, to increase half-life in the body. Thus, the invention includes siRNA derivatives that include siRNA having two complementary strands of nucleic acid, such that the two strands are crosslinked. For example, a 3′ OH terminus of one of the strands can be modified, or the two strands can be crosslinked and modified at the 3′ OH terminus. The siRNA derivative can contain a single crosslink (e.g., a psoralen crosslink). In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acid compositions of the invention can be unconjugated or can be conjugated to another moiety, such as a nanoparticle, to enhance a property of the compositions, for example, a pharmacokinetic parameter such as absorption, efficacy, bioavailability, and/or half-life. The conjugation can be accomplished by methods known in the art, for example, using the methods of Lambert et al. (2001), Drug Deliv. Rev., 47(1), 99-112 (describes nucleic acids loaded to polyalkylcyanoacrylate (PACA) nanoparticles); Fattal et al., J Control Release 53:137-143, 1998 (describes nucleic acids bound to nanoparticles); Schwab et al., Ann. OncoL, 5 Suppl. 4:55-8, 1994 (describes nucleic acids linked to intercalating agents, hydrophobic groups, polycations or PACA nanoparticles); and Godard etal.,Eur.J.Biochem., 232:404410. 1995 (describes nucleic acids linked to nanoparticles).

The nucleic acid molecules of the present invention can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, for example, using ³H, ³²P, or other appropriate isotope.

The ability of the siRNAs of the present invention to mediate RNAi is particularly advantageous considering the rapid mutation rate of viruses. The invention contemplates several embodiments which further leverage this ability by, e.g., targeting a region of the CMV genome that is present in an mRNA that encodes more than one protein. This approach provides the advantage that it allows inhibition of two or more proteins with a single RNAi agent. A second important advantage is that it much less likely that an escape mutant will appear in a region of genomic sequence from which multiple proteins are derived than in a region that encodes a single protein. In an exemplary embodiment, exon 3 of the UL123 and UL122 HCMV genes is targeted, as discussed in greater detail below. Additionally or alternatively, a subject's infected cells can be procured and the genome of the CMV virus within it sequenced or otherwise analyzed to synthesize one or more corresponding RNAi agents, e.g, siRNAs, shRNAs, or plasmids or transgenes expressing siRNAs. Additionally or alternatively, high mutation rates can be addressed by introducing several siRNAs that target different and/or staggered regions of the CMV genome.

Molecules that can be used as “negative controls” will be known to one of ordinary skill in the art. For example, a negative control siRNA can have the same nucleotide composition as the selected siRNA, but without significant sequence complementarity to the appropriate genome. Such negative controls may be designed by randomly scrambling the nucleotide sequence of the selected siRNA; a homology search can be performed to ensure that the negative control lacks homology to any other gene in the appropriate genome. In addition, negative control siRNAs can be designed by introducing a sufficient number of base mismatches into the sequence to limit sequence complementarity (e.g., more than about 4, 5, 6, 7 or more base mismatches).

2. Manufacture of siRNA

In preferred embodiments, siRNAs are synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the siRNA. Inhibition may be targeted by specific transcription in an organ, tissue, or cell type; stimulation of an environmental condition (e.g., infection, stress, temperature, chemical inducers); and/or engineering transcription at a developmental stage or age. A transgenic organism that expresses siRNA from a recombinant construct may be produced by introducing the construct into a zygote, an embryonic stem cell, or another multipotent cell derived from the appropriate organism.

In addition, not only can an siRNA of the invention be used to inhibit expression of more than one protein within the cell, but the siRNAs can be replicated and amplified within a cell by the host cell's enzymes. Alberts, et al., The Cell 452 (4th Ed. 2002). Thus, a cell and its progeny can continue to carry out RNAi even after the CMV RNA has been degraded.

RNA may be produced enzymatically or by partial/total organic synthesis, any modified ribonucleotide can be introduced by in vitro enzymatic or organic synthesis. In one embodiment, a siRNA is prepared chemically. Methods of synthesizing RNA molecules are known in the art, in particular, the chemical synthesis methods as de scribed in Verna and Eckstein, Annul Rev. Biochem. 67:99-134 (1998). In another embodiment, a siRNA is prepared enzymatically. For example, a siRNA can be prepared by enzymatic processing of a long dsRNA having sufficient complementarity to the desired target RNA. Processing of long dsRNA can be accomplished in vitro, for example, using appropriate cellular lysates and ds-siRNAs can be subsequently purified by gel electrophoresis or gel filtration. In an exemplary embodiment, RNA can be purified from a mixture by extraction with a solvent or resin, precipitation, electrophoresis, chromatography, or a combination thereof. Alternatively, the RNA may be used with no or a minimum of purification to avoid losses due to sample processing.

The siRNAs can also be prepared by enzymatic transcription from synthetic DNA templates or from DNA plasmids isolated from recombinant bacteria. Typically, phage RNA polymerases are used such as T7, T3 or SP6 RNA polymerase (Milligan & Uhlenbeck, Methods Enzymol. 180:51-62 (1989)). The RNA may be dried for storage or dissolved in an aqueous solution. The solution may contain buffers or salts to inhibit annealing, and/or promote stabilization of the single strands.

3. siRNA Vectors

Another aspect of the present invention includes a vector that expresses one or more siRNAs that include sequences sufficiently complementary to a portion of the CMV (e.g., HCMV) genome to mediate RNAi. The vector can be administered in vivo to thereby initiate RNAi therapeutically or prophylactically by expression of one or more copies of the siRNAs.

In one embodiment, synthetic shRNA is expressed in a plasmid vector. In another, the plasmid is replicated in vivo. In another embodiment, the vector can be a viral vector, e.g., a retroviral vector. Use of vectors and plasmids are advantageous because the vectors can be more stable than synthetic siRNAs and thus effect long-term expression of the siRNAs.

Viral genomes mutate rapidly and a mismatch of even one nucleotide can, in some instances, impede RNAi. Accordingly, also within the scope of the invention is a vector that expresses a plurality of siRNAs to increase the probability of sufficient homology to mediate RNAi. Preferably, these siRNAs are staggered along the CMV (e.g., HCMV) genome. In one embodiment, one or more of the siRNAs expressed by the vector is a shRNA. The siRNAs can be staggered along one portion of the CMV (e.g., HCMV) genome or target different genes in the CMV (e.g., HCMV) genome. In one embodiment, the vector encodes about 3 siRNAs, more preferably about 5 siRNAS. The siRNAs can be targeted to conserved regions of the CMV (e.g., HCMV) genome.

4. Antisense Oligonucleotides.

An “antisense” nucleic acid can include a nucleotide sequence that is complementary to a “sense” nucleic acid encoding a protein, for example, complementary to the coding strand of a double-stranded cDNA molecule or complementary to an mRNA sequence. The antisense nucleic acid can be complementary to an entire coding stand of a viral, e.g., CMV (e.g., HCMV), gene, or to only a portion thereof.

An antisense nucleic acid can be designed such that it is complementary to the entire coding region of a viral, e.g., HCMV, mRNA, but more preferably is an oligonucleotide that is antisense to only a portion of the coding or noncoding region of a viral, e.g., HCMV, mRNA. For example, the antisense oligonucleotide can be complementary to the region surrounding the translation start site of a viral, e.g., HCMV, mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a viral gene, e.g., an HCMV gene, e.g., to Exon 3 of the genes encoding the IE72, IE86, and IE55 proteins, to thereby inhibit expression of these proteins, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al, Nucleic Acids. Res. 15:6625-6641, 1987). The antisense nucleic acid molecule can also comprise a 2′-o-methylribonucleotide (Inoue et al., ‘Nucleic Acids Res. 15:6131-6148, 1987) or a chimeric RNA-DNA analogue (Inoue et al. FEBS Lett., 215:327-330, 1987).

5. Ribozymes

Ribozymes are a type of RNA that can be engineered to enzymatically cleave and inactivate other RNA targets in a specific, sequence-dependent fashion. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art. A ribozyme having specificity for a viral gene, e.g., a CMV gene, e.g., a IE72, IE86, and IE55-encoding nucleic acid (e.g., Exon 3), can include one or more sequences complementary to, for example, the nucleotide sequence of Exon 3 (i.e., SEQ ID NO:1 or SEQ ID NO:2), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence encoding Exon 3 mRNA. See, e.g., Cech et al. U.S. Pat. No. 4,987,071; and Cech et al U.S. Pat. No. 5,116,742. Alternatively, Exon 3 can be used to select a catalytic RNA having a specific ribonuclease activity from a pool of RNA molecules. See, e.g., Bartel and Szostak, Science 261:1411-1418,1993.

Agents of the invention can be administered alone or in combination to achieve the desired therapeutic result. The invention also contemplates administration with other agents, e.g., antiviral agents, to achieve the desired therapeutic result.

Methods of Introducing RNAs, Vectors, and Host Cells

Physical methods of introducing the agents of the present invention (e.g., siRNAs, vectors, or transgenes) include injection of a solution containing the agent, bombardment by particles covered by the agent, soaking the cell or organism in a solution of the agent, or electroporation of cell membranes in the presence of the agent. A viral construct packaged into a viral particle would accomplish both efficient introduction of an expression construct into the cell and transcription of RNA, including siRNAs, encoded by the expression construct. Other methods known in the art for introducing nucleic acids to cells may be used, such as lipid-mediated carrier transport, chemical-mediated transport, such as calcium phosphate, and the like. Thus the siRNA may be introduced along with components that perform one or more of the following activities: enhance siRNA uptake by the cell, inhibit annealing of single strands, stabilize the single strands, or otherwise increase inhibition of the target gene.

The agents may be directly introduced into the cell (i.e., intracellularly); or introduced extracellularly into a cavity, interstitial space, into the circulation of an organism, introduced orally, or may be introduced by bathing a cell or organism in a solution containing the RNA. Vascular or extravascular circulation, the blood or lymph system, and the cerebrospinal fluid are sites where the agent may be introduced.

Cells may be infected with CMV (e.g., HCMV) upon delivery of the agent or exposed to the CMV (e.g., HCMV) virus after delivery of agent. The cells may be derived from or contained in any organism. The cell may be from the germ line, somatic, totipotent or pluripotent, dividing or non-dividing, parenchyma or epithelium, immortalized or transformed, or the like. The cell may be a stem cell, e.g., a hematopoietic stem cell, or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Depending on the particular target gene and the dose of double stranded RNA material delivered, this process may provide partial or complete loss of function for the target gene. A reduction or loss of gene expression in at least 30%, 40%, 50%, 60%, 70%, 80%, 90%, 95% or 99% or more of targeted cells is exemplary. Inhibition of gene expression refers to the absence (or observable decrease) in the level of viral protein, RNA, and/or DNA. Specificity refers to the ability to inhibit the target gene without manifesting effects on other genes, particularly those of the host cell. The consequences of inhibition can be confirmed by examination of the outward properties of the cell or organism or by biochemical techniques such as RNA solution hybridization, nuclease protection, Northern hybridization, reverse transcription gene expression monitoring with a microarray, antibody binding, enzyme linked immunosorbent assay (ELISA), integration assay, Western blotting, radioimmunoassay (RIA), other immunoassays, and fluorescence activated cell analysis (FACS).

For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT); green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentarnycin, hygromycin, kanamycin, lincomycin, methotrexate, phosphinothricin, puromycin, and tetracyclin. Depending on the assay, quantitation of the amount of gene expression allows one to determine a degree of inhibition which is greater than 10%, 33%, 50%, 90%, 95% or 99% as compared to a cell not treated according to the present invention. Lower doses of injected material and longer times after administration of siRNA may result in inhibition in a smaller fraction of cells (e.g., at least 10%, 20%, 50%, 75%, 90%, or 95% of targeted cells).

Quantitation of gene expression in a cell may show similar amounts of inhibition at the level of accumulation of target RNA or translation of target protein. As an example, the efficiency of inhibition may be determined by assessing the amount of gene product in the cell; RNA may be detected with a hybridization probe having a nucleotide sequence outside the region used for the inhibitory double-stranded RNA, or translated polypeptide may be detected with an antibody raised against the polypeptide sequence of that region.

The siRNA may be introduced in an amount that allows delivery of at least one 30 copy per cell. Higher doses (e.g., at least 5, 10, 100, 500 or 1000 copies per cell) of material may yield more effective inhibition; lower doses may also be useful for specific applications.

Methods of Treatment

The present invention provides for both prophylactic and therapeutic methods for treating a subject at risk of (or susceptible to) or a subject having a virus (e.g., CMV virus, e.g., HCMV). “Treatment”, or “treating” as used herein, is defined as the application or administration of a therapeutic agent (e.g., a siRNA or vector or transgene encoding same) to a patient, or application or administration of a therapeutic agent to an isolated tissue or cell line from a patient, who has a virus with the purpose to cure, heal, alleviate, relieve, alter, remedy, ameliorate, improve or affect the virus, or symptoms of the virus. The term “treatment” or “treating” is also used herein in the context of administering agents prophylactically, e.g., to inoculate against a virus.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, infection with the CMV (e.g., HCMV) virus or a condition associated with the CMV virus, e.g., retinitis, pneumonitis, restenosis, cervical carcinoma, prostate cancer, adenocarcinoma of the colon, disseminated viremia, and organ dysfunction, by administering to the subject a prophylactically effective agent that includes any of the siRNAs or vectors or transgenes discussed herein. Administration of a prophylactic agent can occur prior to the manifestation of symptoms characteristic of CMV infection, such that CMV infection and/or CMV related diseases are prevented.

In a preferred embodiment, the prophylactically effective agent is administered to the subject prior to exposure to the CMV virus. In another embodiment, the agent is administered to the subject after exposure to the CMV virus to delay or inhibit its progression. Thus, the method is prophylactic in the sense that healthy cells are protected from CMV infection. The methods generally include administering the agent to the subject such that CMV replication or infection is prevented or inhibited. Preferably, CMV progeny virus formation is inhibited or prevented. Additionally or alternatively, it is preferable that CMV replication is inhibited or prevented. In one embodiment, the siRNA degrades the CMV RNA transcripts in the early stages of CMV replication, for example, shortly after entry into the cell. In this manner, the agent can prevent healthy cells in a subject from becoming infected. In another embodiment, the siRNA degrades the viral RNA transcripts in the late stages of replication. Any of the strategies discussed herein can be employed in these methods, such as administration of an siRNA targeting an exon present in a viral mRNA that is translated into more than one protein, e.g., an siRNA that targets exon 3 of the UL123 and UL122 genes encoding IE72, IE86 and IE55 proteins. Additionally or alternatively, a vector that expresses a plurality of siRNAs sufficiently complementary to the CMV genome to mediate RNAi can be employed.

2. Therapeutic Methods

Another aspect of the invention pertains to methods of modulating target gene expression, protein expression or activity for therapeutic purposes. Accordingly, in an exemplary embodiment, the modulatory method of the invention involves contacting a cell infected with the virus with a therapeutic agent (e.g., a siRNA or vector or transgene encoding same) that is specific for a portion of the viral genome such that RNAi is mediated. These modulatory methods can be performed ex vivo (e.g., by culturing the cell with the agent) or, alternatively, in vivo (e.g., by administering the agent to a subject). The methods can be performed ex vivo and then the products introduced to a subject (e.g., gene therapy).

The therapeutic methods of the invention generally include initiating RNAi by administering the agent to a subject infected with the virus (e.g., HCMV). The agent can include one or more siRNAs, one or more siRNA complexes, vectors that express one or more siRNAs (including shRNAs), or transgenes that encode one or more siRNAs. The therapeutic methods of the invention are capable of reducing viral production (e.g., viral titer), by about 1-2-fold, 2-4-fold, 4-8-fold, 5-10-fold, 10-20-fold, 30-50-fold, 60-80-fold, 90-fold, 100-fold, 200-fold, 300-fold, 400-fold, 500-fold or 1000-fold.

In a preferred embodiment, infected cells are obtained from a subject and analyzed to determine one or more sequences from the virus genomes present in that subject, siRNA is then synthesized to be sufficiently homologous to mediate RNAi (or vectors are synthesized to express such siRNAs), and delivered to the subject. This approach is advantageous because it addresses the particular virus mutations present in the subject. This method can be repeated periodically, to address further mutations in that subject and/or provide boosters for that subject.

Additionally, the therapeutic agents and methods of the present invention can be used in co-therapy with post-transcriptional approaches (e.g., with ribozymes and/or antisense siRNAs, as described herein).

3. Dual Prophylactic and Therapeutic Method

Also within the scope of the invention, a two-pronged attack on the CMV virus is effected in a subject that has been exposed to the CMV virus. An infected subject can thus be treated both prophylactically and therapeutically, such that the agent prevents infection by degrading virally encoded transcripts during early stages of replication.

One skilled in the art can readily determine the appropriate dose, schedule, and method of administration for the exact formulation of the composition being used, in order to achieve the desired “effective level” in the individual patient. One skilled in the art also can readily determine and use an appropriate indicator of the “effective level” of the compounds of the present invention by a direct (e.g., analytical chemical analysis) or indirect (e.g., with surrogate indicators of viral infection) analysis of appropriate patient samples (e.g., blood and/or tissues).

The prophylactic or therapeutic pharmaceutical compositions of the present invention can contain other pharmaceuticals, in conjunction with a vector according to the invention, when used to treat CMV infection therapeutically. These other pharmaceuticals can be used in their traditional fashion (i.e., as agents to treat CMV infection). Representative examples of these additional pharmaceuticals that can be used include antiviral compounds, immunomodulators, immunostimulants, antibiotics, and other agents and treatment regimes (including those recognized as alternative medicine) that can be employed to treat CMV-associated conditions (e.g., retinitis, pneumonitis, restenosis, cervical carcinoma, prostate cancer, adenocarcinoma of the colon, disseminated viremia, and organ disfunction). Antiviral compounds include, but are not limited to, ddI, ddC, gancylclovir, fluorinated dideoxynucleotides, nonnucleoside analog compounds such as nevirapine (Shih, et al., PNAS 88: 9978-9882 (1991)), TIBO derivatives such as R82913 (White, et al., Antiviral Research 16: 257-266 (1991)), and BI-RJ-70 (Shih, et al., Am. J Med. 90 (Suppl. 4A): 8S-17S (1991). Immunomodulators and immunostimulants include, but are not limited to, various interleukins, CD4, cytokines, antibody preparations, blood transfusions, and cell transfusions. Antibiotics include, but are not limited to, antifungal agents, antibacterial agents, and anti-Pneumocystis carinii agents.

A siRNA or vector according to the invention can be delivered to cells cultured ex vivo prior to reinfusion of the transfected cells into the patient or in a delivery vehicle complex by direct in vivo injection into the patient or in a body area rich in the target cells. The in vivo injection may be made subcutaneously, intravenously, intramuscularly or intraperitoneally. Techniques for ex vivo and in vivo gene therapy are known to those skilled in the art. Generally, the compositions are administered in a manner compatible with the dosage formulation, and in such amount as will be prophylactically and/or therapeutically effective. The quantity to be administered depends on the subject to be treated, including, e.g., whether the subject has been exposed to CMV or infected with CMV, or is afflicted with a CMV-associated condition, and the degree of protection desired. Suitable regimens for initial administration and booster shots are also variable but are typified by an initial administration followed by subsequent inoculations or other administrations. Precise amounts of active ingredients required to be administered depend on the judgment of the practitioner and may be peculiar to each subject. It will be apparent to those of skill in the art that the therapeutically effective amount of a composition of this invention will depend upon the administration schedule, the unit dose of agent (e.g., siRNA, vector and/or transgene) administered or expressed by an expression vector that is administered, whether the compositions are administered in combination with other therapeutic agents, the immune status and health of the recipient, and the therapeutic activity of the particular nucleic acid molecule, delivery complex, or ex vivo transfected cell.

4. Pharmacogenomics

The prophylactic and/or therapeutic agents (e.g., a siRNA or vector or transgene encoding same) of the invention can be administered to treat (prophylactically or therapeutically) individuals infected with a virus such as a virus of the herpesviridae family (e.g., CMV, e.g., HCMV). In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a therapeutic agent as well as tailoring the dosage and/or therapeutic regimen of treatment with a therapeutic agent.

Pharmacogenomics deals with clinically significant hereditary variations in the response to drugs due to altered drug disposition and abnormal action in affected persons. See, for example, Eichelbaum, M., et al., Clin. Exp. Pharmacol. Physiol. 23(10-11): 983-985 (1996) and Linder, M. W., et al., Clin. Chem. 43(2):254-266 (1997). In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a target gene polypeptide of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a therapeutic agent of the present invention can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a therapeutic agent, as described herein.

Therapeutic agents can be tested in an appropriate animal model. For example, a siRNA (or expression vector or transgene encoding same) as described herein can be used in an animal model to determine the efficacy, toxicity, or side effects of treatment with said agent. Alternatively, a therapeutic agent can be used in an animal model to determine the mechanism of action of such an agent. For example, an agent can be used in an animal model to determine the efficacy, toxicity, or side effects of treatment with such an agent. Alternatively, an agent can be used in an animal model to determine the mechanism of action of such an agent.

Pharmaceutical Compositions and Methods of Administration

The siRNA molecules of the invention can be incorporated into pharmaceutical compositions. Such compositions typically include the nucleic acid molecule and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral (e.g., intravenous), intradermal, subcutaneous, oral (e.g., inhalation), transdermal (topical), transmucosal, rectal, ocular (topical), and intraocular injection (e.g., intravitreal injection) administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, ParsippanyrNJ)‘or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, for example, gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

The compounds can also be administered by transfection or infection using methods known in the art, including but not limited to the methods described in McCaffrey et al, Nature 418:38-39, 2002 (hydrodynamic transfection); Xia et al, Nature Biotechnol, 20:1006-1010, 2002 (viral-mediated delivery); or Putnam, Am. J Health Syst. Pharm. 53:151-160, 1996, erratum at Am. J Health Syst. Pharm. 53:325, 1996).

The compounds can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable mtcroparticle delivery systems can also be used (e.g., as described in U.S. Pat. No. 6,471,996).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a nucleic acid molecule (i.e., an effective dosage) depends on the nucleic acid selected. For instance, if a plasmid encoding shRNA is selected, single dose amounts in the range of approximately 1 μg to 10000 mg may be administered; in some embodiments, 10, 30, 100 or 1000 μg maybe administered. In some embodiments, 1 g of the compositions can be administered. The compositions can be administered from one or more times per day to one or more times per week, including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

The nucleic acid molecules of the invention can be inserted into expression constructs, e.g, viral vectors, retro viral vectors, expression cassettes, or plasmid viral vectors, e.g., using methods known in the art, including but not limited to those described in Xia et al., (2002), supra Expression constructs can be delivered to a subject by, for example, inhalation, orally, intravenous injection, local administration (see U.S. Pat. 5,328,470) or by stereotactic injection (see, e.g, Chen et al (1994), Proc. Natl. Acad. Sci. USA, 91, 3054-3057). The pharmaceutical preparation of the delivery vector can include the vector in an acceptable diluent, or can comprise a slow release matrix in which the delivery vehicle is imbedded. Alternatively, where the complete delivery vector can be produced intact from recombinant cells, e.g., retroviral vectors, the pharmaceutical preparation can include one or more cells which produce the gene delivery system.

The nucleic acid molecules of the invention can also include small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′UU-overhangs; subsequently, the ends of these shRNAs are processed, converting the shRNAs into siRNA-like molecules of-about-21 nucleotides. (See, Brummelkamp et al. (2002), Science, 296, 550-553; Lee et al, (2002); Miyagishi and Taira (2002), Nature Biotechnol., 20, 497-500; Paddison et al. (2002), Genes Dev., 16, 948-958; Paul et al. (2002), Nature Biotechnol., 20, 505-508; Sui et al. (2002), Proc. Natl. Acad. Sci. USA, 99(6), 5515-5520; Yu et al. (2002), Proc. Natl. Acad. Sci. USA, 99(9), 6047-6052.) More information about shRNA design and use may be found at the internet addresses: katahdin.cshl.org:9331/RNAi/docs/BseRI-BamHI_Strategy.pdf and katahdin.cshl.org:9331/RNAi/docs/Web_version_of PCR_strategyl.pdf.

The expression constructs may be any construct suitable for use in the appropriate expression system and include, but are not limited to retroviral vectors, linear expression cassettes, plasmids and viral or virally-derived vectors, as known in the art. Such expression constructs may include one or more inducible promoters, RNA Pol III promoter systems such as U6 snRNA promoters or HI RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct. (Tuschl, T. (2002), Nature Biotechnol., 20, 440-448).

The pharmaceutical compositions can be included in a container, pack, or dispenser together with instructions for administration.

Knockout and/or Knockdown Cells or Organisms

A further preferred use for the siRNAs of the present invention (or vectors or transgenes encoding that subsequently express siRNAs in the cell) is a functional analysis to be carried out in CMV (e.g., HCMV) eukaryotic cells, or eukaryotic non-human organisms, preferably mammalian cells or organisms and more preferably human cells, e.g. cell lines such as HeLa or 293 or rodents, e.g rats and mice. The cells may be infected with CMV (e.g., HCMV) virus or subsequently infected. The siRNAs, vectors or transgenes can be any of the agents discussed herein, e.g., a vector that expresses one or more shRNAs that target one or more portions of the CMV (e.g., HCMV) genome.

By administering a suitable siRNA molecule or molecules which are sufficiently homologous to a target portion of the CMV (e.g., HCMV) genome to mediate RNA interference, a specific knockout or knockdown phenotype can be obtained in a target cell, e.g. in cell culture or in a target organism.

Gene-specific knockout or knockdown phenotypes of cells or non-human organisms, particularly of human cells or non-human mammals may be used in analytic to procedures, e.g., in the functional and/or phenotypical analysis of complex physiological processes such as analysis of gene expression profiles and/or proteomes. Preferably the analysis is carried out by high throughput methods using oligonucleotide based chips.

This invention is further illustrated by the following examples that should not be construed as limiting. The contents of all references, patents and published patent applications cited throughout this application are incorporated herein by reference.

### EXAMPLES

The following materials, methods, and examples are illustrative only and not intended to be limiting.

### Experimental Procedures for Examples 1-3

Preparation of HCMV-Infected Cells.

HCMV Towne strain (passage 37) or HCMV AD169 strain (American Type Culture Collection) was propagated in HEL fibroblasts and virus stocks prepared as described in Huang (J. Virol. 16:298-310, 1975). Cultures of human endometrial stromal cells were established as described in Dorman et a/., In Vitro 18:919-928, 1982. Essentially, endometrial tissue from hysterectomy specimens was fragmented and dispersed by incubation with collagenase. The resulting cells were plated and cultured in a 1:1 mixture of RPMI1640 and Opti-MEM (Gibco) supplemented with 10% fetal calf serum, 2 μg/mL of insulin, 4 mM glutamine, and penicillin-streptomycin. Two cell types were initially plated: the major species being the stromal cell type and a minor component of epithelial cells. The epithelial cells were lost by two passages of culturing in the presence of serum. The remaining cell type was defined as endometrial stromal cells by its responsiveness to estrogen including the induced secretion of collagen and laminin and its growth inhibition by IL-1. An immortalized endometrial stromal cell was created by transfecting origin-deficient SV40 DNA (ori-tsA209 SV40) containing a temperature sensitive large T gene into cells by electroporation and has been characterized elsewhere for large Tₜₛ function and inactivation at the permissive and nonpermissive temperatures, respectively (Rinehart et al, Carcinogenesis 14:993-999, 1993). Immortalized stromal cells were cultured at the permissive temperature for large T function (33° C.) and shifted to the nonpermissive temperature (39° C.) as needed. All cultures were infected with HCMV at an MOI of 5. Other cells, such as normal human fibroblasts, can be infected in this same manner.

Synthetic RNA Oligo/Duplex Processing.

There are several options for the custom synthesis of siRNA oligonucleotides and presynthesized siRNA duplexes. One option is a water-soluble, stable, 2′-protected RNA, which is readily deprotected in aqueous buffers upon receipt from the supplying company (the RNA molecules of the invention can be water-soluble, 2′-protected RNAs). The 2′-protection helps ensure the RNA is not degraded before use. The pair of RNA oligonucleotides can be simultaneously 2′-deprotected and annealed in the same reaction as a further precaution against degradation. The siRNA duplex can then be readily desalted via ethanol precipitation directly from the aqueous 2′-deprotection/annealing reaction. After deprotection and annealing, the RNA pellet is resuspended in 400 μl buffer. To ethanol precipitate the RNA, the solution is adjusted to 0.3 M NaCl by addition of 26 μl 5 M NaCl. Finally, 1500 μl of absolute ethanol is added and the mixture is vortexed. The sample is incubated for 1 to 2 hours on dry ice or at −20 ° C., and the RNA pellet is collected by centrifugation. All liquid is removed and the pellet is re-dissolved in 200-400 μl of sterile water. The RNA concentration is determined by UV spectroscopy (1 A260-unit is equivalent to 32 μg RNA) followed by annealing (see below). It should be noted that the crude RNA products are more than 85% full-length, which makes gel-purification of siRNAs for knockdown applications unnecessary.

Alternatively, the RNA is provided fully deprotected, desalted and aliquotted in 50 nanomole amounts. The commercially-provided RNA is dissolved in water followed by siRNA annealing (see below). In another option, the siRNAs is provided as a purified duplex with a purity >97%. The commercially obtained RNA duplex pellet is dissolved in water and is used directly for transfection (see below). It is also possible to order RNA duplexes properly formed and ready for transfection. The siRNAs utilized in the experiments described herein included, but are not limited to, siRNAs of SEQ ID NO:1 and SEQ ID NO:2 and their respective complementary sequences. siRNAs with the sequences SEQ ID NO:1 and SEQ ID NO:2 include 5′PO₄ groups and -dTdT at the 3′ends.

RNAi oligonucleotides can be annealed according to standard protocols known in the art, e.g., according to the directions of the manufacturer. For example, 20 μM single-stranded 21-nt RNAs in annealing buffer (100 mM potassium acetate, 30 mM HEPES-KOH at pH 7.4, 2 mM magnesium acetate) can be incubated for one minute at 90 ° C., followed by one hour at 37° C. The solution can then be stored frozen at −20° C. and freeze-thawed multiple times. Successful duplex formation can be confirmed by examining the siRNA duplexes using 20% non-denaturing polyacrylamide gel electrophoresis (PAGE). RNAi duplexes can then be transfected into cells that are cultured according to the manufacturer's directions or can be formulated into a pharmaceutical composition as described above.

siRNA Delivery for Longer-Term Expression.

Synthetic siRNAs can be delivered into cells by cationic liposome transfection and electroporation. However, these exogenous siRNA only show short-term persistence of the silencing effect (4-5 days). Several strategies for expressing siRNA duplexes within cells from recombinant DNA constructs allow longer-term target gene suppression in cells, including mammalian Pol III promoter systems (e.g., HI or U6/snRNA promoter systems (Tuschl, supra) capable of expressing functional double-stranded siRNAs; (Bagella et al. J. Cell Physiol. 177:206-213, 1998; Lee et al, supra; Miyagishi et al., supra; Paul et al., supra; Yu et al., supra; Sui et al., supra). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by HI or U6 snRNA promoter and expressed in cells, can inhibit target gene expression (Bagella et al., supra; Lee et al., supra; Miyagishi et al., supra; Paul et al. (2002), supra; Yu et al. (2002), supra; Sui et al. (2002) supra). Constructs containing siRNA sequence under the control of the T7 promoter also make functional siRNAs when cotransfected into the cells with a vector expressing T7 RNA polymerase (Jacque, supra). Accordingly, the dsRNAs, siRNAs or other inhibitory nucleic acids of the present invention can be expressed under the control of the HI, U6, T7, or similar promoters.

Animal cells express a range of noncoding RNAs of approximately 22 nucleotides termed micro RNA (miRNAs) which can regulate gene expression at the post transcriptional or translational level during animal development. miRNAs are excised from an approximately 70-nucleotide precursor RNA stem-loop, probably by Dicer, an RNase III-type enzyme, or a homolog thereof. By substituting the stem sequences of the miRNA precursor with an miRNA sequence complementary to the target mRNA, a vector construct which expresses the novel miRNA can be used to produce siRNAs to initiate RNAi against specific mRNA targets in mammalian cells (Zeng, supra). Accordingly, miRNAs that target viral sequences (e.g., herpesvirus sequences such as HCMV sequences) are within the scope of the present invention. When expressed by DNA vectors containing polymerase III promoters, micro-RNA designed hairpins are active in silencing gene expression (McManus, supra). Viral-mediated delivery mechanisms can also be used to induce specific silencing of targeted genes through expression of siRNA, for example by generating recombinant adenoviruses harboring siRNA under RNA Pol II promoter transcription control (Xia et al, supra). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression (Id.). In an animal model, whole-embryo electroporation can efficiently deliver synthetic siRNA into post-implantation mouse embryos (Calegari et al, Proc. Natl. Acad Sci. USA, 99:14236-14240, 2002). In adult mice, efficient delivery of siRNA can be accomplished by “high-pressure” delivery technique, a rapid injection (within 5 seconds) of a large volume of siRNA-containing solution into the animal via the tail vein (Liu, supra; McCaffrey, supra; Lewis, Nature Genetics 32:107-108, 2002). Nanoparticles and liposomes can also be used to deliver siRNA into animals.

### Example 1

### In Vitro RNAi Suppression of HCMV IE Expression

Diploid human fibroblasts, the model cell type known in the art used for HCMV research, were electroporated with control of HCMV IE specific siRNAs. These siRNAs target Exon 3, an exon shared by both UL122 and UL123 genes and shared by the open reading frames for expression of IE72, IE86, and IE55. Electroporation of human fibroblasts yielded >90% transfection efficiency of surviving cells. Fibroblasts were infected 24 hours later with recombinant adenoviruses expressing UL123 encoding 1E1 (also known as IE72) or UL122 encoding IE2 (also known as IE86). At 48 hpi, cell lysates were generated and IE gene expression was examined by immunoblotting with an antibody specific for an epitope shared by 1E1 and 1E2. In FIG. 3, IE72, termed 1E1 in the figure, was expressed at very high levels with a recombinant adeno virus and targeted for RNAi with the IEX3 and IEY3 X3 which refers to IEX3, SEQ ID NO:1, and Y3 which refers to SEQ ID NO:2. IE expression is greatly reduced in the presence of X3 siRNA whereas a more modest but significant reduction is seen upon treatment with Y3. The levels of IE expression in the “pum” lane exceed that achieved by high MOI infections with HCMV.

RNAi-Mediated Reduction of Reporter Gene Expression.

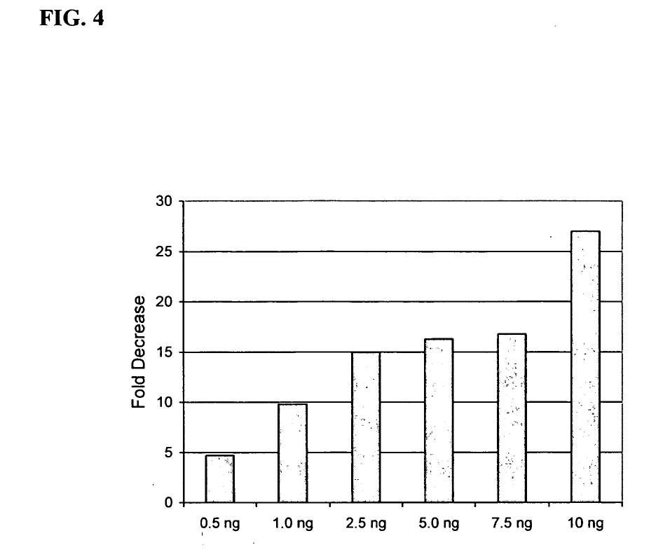

Reduction of reporter gene expression by transfection of small interfering RNAs (siRNAs) was also examined, and the results are shown in FIG. 4. FIG. 4 is a bar graph with the y-axis showing the fold decrease in luciferase activity. The x-axis represents the varying concentrations of siRNA applied to the COS cells. COS cells were co-transfected with firefly luc and Renilla luc expressing plasmids along with siRNA to firefly luc or control siRNA (to Drosophila pumilio). Lysates were generated and luciferase activity determined. Samples were normalized to Renilla luciferase activity and plotted as fold inhibition relative to transfections with control siRNAs. Similar results were obtained in HeLa and 293 cells targeting firefly luc or GFP with gene-specific siRNAs.

### Example 2

### RNAi Supression of HCMV IE Gene Expression During HCMV Replication

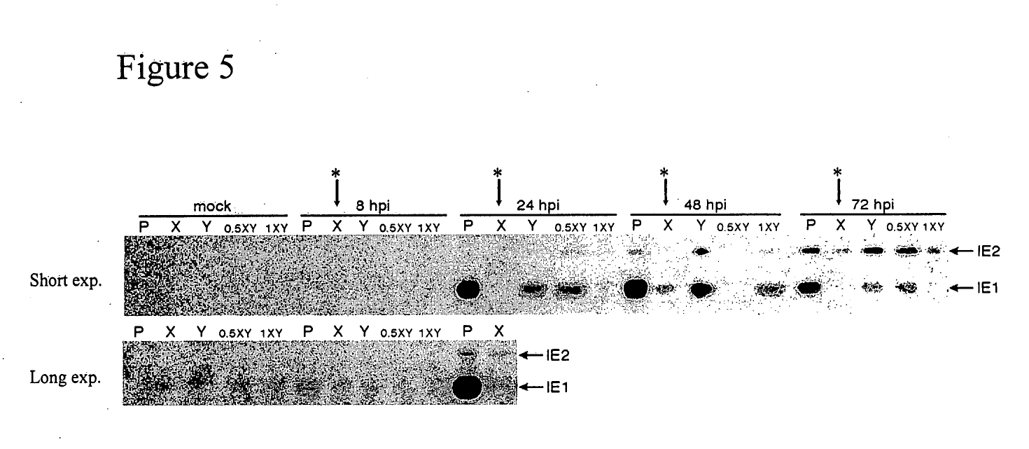

Diploid human fibroblasts were electroporated with control siRNA (labeled “P” in Figure) or with the HCMV-specific siRNAs (IEX3, labeled “X”, and EIY3, labeled “Y”, in FIG. 5) as described above in Example 1 and FIG. 3. In addition, combinations of the HCMV-specific siRNAs IEX3 (“X”) and IEY3 (“Y”) were co-electroporated where indicated. As noted in FIG. 5, “0.5XY” denotes a mixture of IEX3 and IEY3 siRNAs wherein the equivalent of one half the amount of siRNA used in electroporations with single siRNA species were used of each siRNA in the combination. In FIG. 5, “1XY” denotes a that an equivalent amount was co-electroporated of each siRNA as was used in electroporations with single siRNA species. At twenty fours hours post transfection, fibroblasts were infected with the HCMV AD169 strain at an MOI of 3. At various times following infection (hours post infection (hpi) in FIG. 5), cell lysates were generated and IE gene expression was examined by immunoblotting according to the methods described in Example 2.

The results of this experiment are presented in FIG. 5, wherein the lower image is a longer exposure of the immunoblotting reaction showing IE1 gene expression at 8 hpi. The results show that at each time point, IE1 and IE2 gene expression was reduced to undetectable or barely detectable levels in samples treated with IEX3 (as indicated by arrow and asterisk in FIG. 5). Another siRNA, IEY3, also reduced IE1 and IE2 gene expression, albeit to a lesser degree than IE1. When various combinations of IEX3 and IEY3 were co-transfected into the cells, IE1 and IE2 gene expression was also reduced, although the effect was less dramatic than with IEX3 alone. These results clearly demonstrated that expression of both IE1 and IE2 genes were targeted by the IEX3 and IEY3 siRNAs.

RNAi Suppression of HCMV IE Gene Expression Results in Suppression of Glycoprotein B Protein Expression During HCMV Infections.

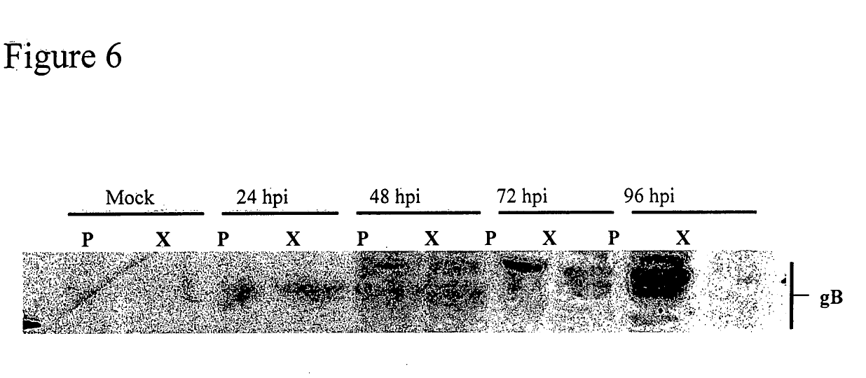

Glycoprotein B (gB) protein is the product of the HCMV late gene, UL55, and is a component of the virion envelope necessary for virus attachment and entry into cells. The effect of RNAi suppression of HCMV IE gene expression was next examined. Diploid human fibroblasts were electroporated with either the Pum or IEX3 siRNAs (labeled as P and X, respectively, in FIG. 6) and then infected with HCMV as described above and in FIG. 5. Cell lysates were generated at the times indicated following viral infection and analyzed for glycoprotein B expression by immunoblotting using an antibody specific for glycoprotein B. Like many glycosylated proteins, glycoprotein B appears as multiple species in immunoblots. The results of this experiment are presented in FIG. 6. At each time point, introduction of IEX3 resulted in reduced levels of glycoprotein B protein. Since IE gene expression is required for transcription of many HCMV late genes, including UL55, the reduction in glycoprotein B levels by IEX3 is likely due to suppression of IE1 and IE2 gene expression by IEX3. Reduced levels of glycoprotein B is also likely to result in lower titers of infectious virus.

RNAi Suppression of HCMV IE Gene Expression Results in Reduced Yields of Progeny Virus.

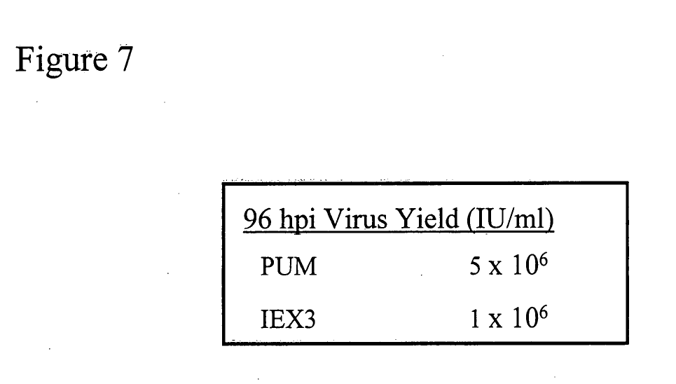

Diploid human fibroblasts were electroporated with siRNAs and infected with HCMV as described above and in FIG. 6. At 96 hpi, culture media containing progeny virus were assayed for virus titer by using a standard infectious center assay. The results, presented in FIG. 7 as infectious units/ml (IU/ml), showed that IEX3 reduced HCMV titers by five fold. Importantly, these results demonstrated that RNAi-mediated suppression of HCMV gene expression inhibited virus replication.

### Example 3

### Efficacy of RNAi Activation in Limiting HCMV Gene Expression and Replication.

To determine the broad-range efficacy of siRNAs in blocking HCMV gene expression, siRNAs can be synthesized against viral genes of each temporal expression class: immediate early (IE), early (E) and late (L). Inhibitory RNAs that target genes in each of these classes and methods in which those RNAs are used to inhibit viral proliferation are within the scope of the present invention. IE gene products induce the expression of E and L genes. Deletion of IE1 is known to result in a greatly attenuated virus that can only replicate at high MOIs, while IE2 is essential for viral replication. The E gene products are responsible for DNA replication and are the targets of traditional antiviral therapeutics. The L gene products are involved in virion maturation and cell-to-cell spread. UL97 is a kinase that activates ganciclovir (GCV). Reducing UL97 expression would be expected to render HCMV resistant to GCV. UL97 is a nonessential gene that will also be used as a target viral gene in the screen for anti-RNAi activity. Table 1 lists the genes that can be targeted and the anticipated outcomes.

TABLE II
Genes to be targeted with siRNAs.

| | | | | Predicted |
|---|---|---|---|---|
| Gene | | | | effect on virus |
| class | Gene name | Protein | Function | replication |
| IE | UL123 | IE1 | Transactivation | Reduction |
| IE | UL122 | IE2 | Transactivation | Reduction/inhibition |
| IE | UL123/UL122 | IE1/IE2 | Transactivation | Inhibition (X3 & Y3 |
| | (exon 3) | | | siRNAs shown in |
| | | | | Preliminary Studies) |
| E | UL54 | Pol | DNA polymerase | Reduction/inhibition |
| E | UL97 | ppUL97 | Phosphorylates | No effect on |
| | | | GCV | replication Induce |
| | | | | resistance to |
| | | | | ganciclovir (GCV) |
| L | UL80 | Protease | Scaffold protease | Reduction/inhibition |
| L | UL55 | gB | Attachment | Reduction/inhibition |
| | | | protein | at low MOI |
| | | | | (reduced cell-to-cell |
| | | | | spread) |
| L | UL75 | gH | Attachment | Reduction/inhibition |
| | | | protein | at low MOI |
| | | | | (reduced cell-to-cell |
| | | | | spread) |

The siRNAs of the invention can be electroporated into fibroblasts under conditions optimized using fluorescently tagged siRNAs (see above). Dose curves of siRNAs can be used to determine optimal conditions for inhibition of gene expression. Viral gene expression can be monitored by northern and immunoblot analysis. Virus replication can be monitored by using a standard plaque assay or infectious center assay at low and high MOIs and the EC₅₀ calculated for each siRNA.

Many virus populations can accumulate mutant forms that are resistant to negative selection pressures. Given the possibility that mutations could result in resistance to an siRNA, attempts can be made to select for escape mutants by treating HCMV infections with low levels of siRNAs through repeated passage in cells. Efforts can be focused on selecting for escape mutants in the DNA polymerase gene (UL54) and the protease gene (UL80) since escape mutants have been observed for these genes when treated with conventional antiviral therapies (Gilbert et al, Drug Res. Updates 5:88-114, 2002). The target gene of any escape mutant can be sequenced to determine if the mutation(s) arise in the region homologous to the siRNA.

A number of embodiments of the invention have been described. Nevertheless, it will be understood that various modifications may be made without departing from the spirit and scope of the invention. Accordingly, other embodiments are within the scope of the following claims.

What is claimed is:

1. A method of inhibiting a cytomegalovirus (CMV), the method comprising exposing a cell infected with CMV to a small inhibitory RNA molecule (siRNA) that targets a CMV gene, under conditions that permit induction of ribonucleic acid interference (RNAi), such that CMV is inhibited.

2. The method of claim 1, wherein the siRNA targets a CMV immediate early gene.

3. The method of claim 1, wherein the siRNA targets a CMV early gene.

4. The method of claim 1, wherein the siRNA targets a CMV late gene.

5. The method of claim 1, wherein the siRNA is a double stranded RNA (dsRNA) molecule, each strand of which is about 18-29 nucleotides long.

6. The method of claim 5, wherein the dsRNA has a 3′dTdT sequence and a 5′ phosphate group (PO4).

7. The method of claim 5, wherein each strand of the dsRNA is encoded by a sequence contained within an expression vector.

8. A method of inhibiting the expression of two or more proteins simultaneously, the method comprising:
(a) providing an siRNA that targets a single mRNA that is translated into the two or more proteins; and
(b) exposing the single mRNA to the siRNA under conditions that permit induction of RNAi, the RNAi inhibiting the single mRNA that is translated into the two or more proteins;
such that expression of the two or more proteins is simultaneously inhibited.

9. The method of claim 8, wherein the siRNA is a double stranded RNA (dsRNA) molecule, each stand of which is about 18-29 nucleotides long.

10. The method of claim 9, wherein each strand of the dsRNA is encoded by a sequence contained within an expression vector.

11. The method of claim 8, wherein the mRNA is expressed from exon 3, exon 2, or exon 1 of UL123 and UL122 genes.

12. A method of using post-transcriptional inhibition to inhibit expression of more than
one protein with a single agent, the method comprising:
(a) providing an RNAi agent capable of targeting an exon that is present in mRNA that is translated into more than one protein; and
(b) administering the RNAi agent to cells in which viral expression is to be inhibited;
such that expression of more than one protein is inhibited by the RNAi agent.

13. The method of claim 12, wherein the exon is exon 3 of genes encoding IE72, IE86, and IE55 proteins.

14. The method of claim 12, wherein the RNAi agent is dsRNA which is greater than about 18 nucleotides and less than about 29 nucleotides in length.

15. The method of claim 12, wherein the RNAi agent is an expression vector expressing dsRNA which is greater than about 18 nucleotides and less than about 29 nucleotides in length.

16. A method of inhibiting viral replication, the method comprising targeting an isolated nucleic acid to an mRNA from which more than one protein involved in viral replication is expressed, such that viral replication is inhibited.

17. The method of claim 12, wherein the mRNA is expressed from exon 3, exon 2, or exon 1 of UL123 and UL122 genes.

18. The method of claim 17, wherein the mRNA expresses two or more of IE72, IE86, and IE55 of CMV.

19. An isolated nucleic acid comprising the sequence of SEQ ID No. 1 or its complement.

20. The isolated nucleic acid of claim 19, wherein T is replaced by U.

21. The isolated nucleic acid of claim 19, wherein the isolated nucleic acid is double-stranded.

22. The isolated nucleic acid of claim 21, wherein the isolated nucleic acid has 3′dTdT and 5′-PO₄.

23. An isolated nucleic acid comprising the sequence of SEQ ID No. 2 or a complement thereof.

24. The isolated nucleic acid of claim 23, wherein T is replaced by U.

25. The isolated nucleic acid of claim 23, wherein the isolated nucleic acid is double-stranded.

26. The isolated nucleic acid of claim 25, wherein the isolated nucleic acid has 3′dTdT and 5′-PO₄.

27. An RNAi agent which is targeted to a CMV nucleic acid encoding one or more CMV proteins.

28. An RNAi agent which is targeted to a CMV nucleic acid encoding one or more of the group consisting of 1E1, 1E2, DNA polymerase, a scaffold protease, gB, and gH.

29. The RNAi agent of claim 28, wherein the RNAi agent consists of dsRNA which is greater than about 18 nucleotides and less than about 29 nucleotides in length.

30. The RNAi agent of claim 29, wherein the dsRNA has 3′dTdT and 5′-PO₄.

31. A vector comprising the sequence of SEQ ID No. 1 and/or SEQ ID NO:2 or a complement thereof.

32. The vector of claim 31, wherein T is replaced by U.

33. The vector of claim 31, wherein the vector is a plasmid vector or a viral vector.

34. The vector of claims 31, 32, or 33, wherein the vector expresses dsRNA greater than about 18 nucleotides and less than about 29 nucleotides in length.

35. The vector of claim 34, wherein the dsRNA has 5′ PO₄ and 3′ TT or 3′dTdT.

36. A host cell comprising the isolated nucleic acid selected from the group consisting of claims 19-26, the RNAi agent selected from the group consisting of claims 27-30, or the vector selected from the group consisting of claims 31-34.

37. The host cell of claim 36, wherein the host cell is infected with CMV.

38. A pharmaceutical composition comprising the isolated nucleic acid selected from the group consisting of claims 19-26, the RNAi agent selected from the group consisting of claims 27-30, or the vector selected from the group consisting of claims 31-34, and a pharmaceutically acceptable carrier.

39. A method of treating a condition associated with CMV infection comprising administering the pharmaceutical composition of claim 38 to a vertebrate mammal with the condition, such that the condition associated with CMV infection is treated.

40. The method of claim 39, wherein the vertebrate mammal is a human patient.

41. The method of claim 39, wherein the vertebrate animal is a non-human primate.

42. The method of claim 39, wherein the CMV-associated condition is one of the group consisting of retinitis, pneumonitis, restenosis, cervical carcinoma, prostate cancer, adenocarcinoma of the colon, disseminated viremia, and organ dysfunction.

43. The method of claim 39, wherein the administering is localized or tissue-specific.

44. The method of claim 43, wherein the CMV-associated condition is retinitis and the administering is by intravitreal injection.


SEQUENCE LISTING (183 sequences)

SEQ ID NO: 1 - DNA, 19 bp, Cytomegalovirus
  siRNA target sequence
ctatgttgaggaaggaggt

SEQ ID NO: 2 - DNA, 19 bp, Cytomegalovirus
  siRNA target sequence
gaaagatgtcctggcagaa

SEQ ID NO: 3 - DNA, 19 bp, Cytomegalovirus
  siRNA target sequence
cgacgttcctgcagactat

SEQ ID NO: 4 - DNA, 19 bp, Cytomegalovirus
  siRNA target sequence
tgttgaggaaggaggttaa

SEQ ID NO: 5 - DNA, 19 bp, Cytomegalovirus
  siRNA target sequence
ggaaggaggttaacagtca

SEQ ID NO: 6 - DNA, 19 bp, Cytomegalovirus
  siRNA target sequence
caagtgaccgaggattgca

SEQ ID NO: 7 - DNA, 19 bp, Cytomegalovirus
  siRNA target sequence
gaactcgtcaaacagatta

SEQ ID NO: 8 - DNA, 19 bp, Cytomegalovirus
  siRNA target sequence
actcgtcaaacagattaag

SEQ ID NO: 9 - DNA, 19 bp, Cytomegalovirus
  siRNA target sequence
ctcgtcaaacagattaagg

SEQ ID NO: 10 - DNA, 19 bp, Cytomegalovirus
  siRNA target sequence
tggtgcggcatagaatcaa

SEQ ID NO: 11 - DNA, 19 bp, Cytomegalovirus
  siRNA target sequence
gacggaagagaaattcact

SEQ ID NO: 12 - DNA, 19 bp, Cytomegalovirus
  siRNA target sequence
gaaattcactggcgccttt

SEQ ID NO: 13 - DNA, 19 bp, Cytomegalovirus
  siRNA target sequence
aattcactggcgcctttaa

SEQ ID NO: 14 - DNA, 19 bp, Cytomegalovirus
  siRNA target sequence
attcactggcgcctttaat

SEQ ID NO: 15 - DNA, 19 bp, Cytomegalovirus
  siRNA target sequence
ttcactggcgcctttaata

SEQ ID NO: 16 - DNA, 19 bp, Cytomegalovirus
  siRNA target sequence
gcctttcgaggagatgaag

SEQ ID NO: 17 - DNA, 19 bp, Cytomegalovirus
  siRNA target sequence
cattgtacctgaggataag

SEQ ID NO: 18 - DNA, 19 bp, Cytomegalovirus
  siRNA target sequence
ttaaggagctgcatgatgt

SEQ ID NO: 19 - DNA, 19 bp, Cytomegalovirus
  siRNA target sequence
aggatgaacttaggagaaa

SEQ ID NO: 20 - DNA, 19 bp, Cytomegalovirus
  siRNA target sequence
acttaggagaaagatgatg

SEQ ID NO: 21 - DNA, 19 bp, Cytomegalovirus
  siRNA target sequence
cttaggagaaagatgatgt

SEQ ID NO: 22 - DNA, 19 bp, Cytomegalovirus
  siRNA target sequence
tttatggatatcctcacta

SEQ ID NO: 23 - DNA, 19 bp, Cytomegalovirus
  siRNA target sequence
aacaatgtgtaatgagtac

SEQ ID NO: 24 - DNA, 19 bp, Cytomegalovirus
  siRNA target sequence
atgagtacaaggtcactag

SEQ ID NO: 25 - DNA, 19 bp, Cytomegalovirus
  siRNA target sequence
tgagtacaaggtcactagt

SEQ ID NO: 26 - DNA, 19 bp, Cytomegalovirus
  siRNA target sequence
gtgacgcttgtatgatgac

SEQ ID NO: 27 - DNA, 19 bp, Cytomegalovirus
  siRNA target sequence
agcggcctctgataaccaa

SEQ ID NO: 28 - DNA, 19 bp, Cytomegalovirus
  siRNA target sequence
accaagcctgaggttatca

SEQ ID NO: 29 - DNA, 19 bp, Cytomegalovirus
  siRNA target sequence
atcatgccggtatcgattc

SEQ ID NO: 30 - DNA, 19 bp, Cytomegalovirus
  siRNA target sequence
aaaccacgcgtcctttcaa

SEQ ID NO: 31 - DNA, 19 bp, Cytomegalovirus
  siRNA target sequence
aaccacgcgtcctttcaag

SEQ ID NO: 32 - DNA, 19 bp, Cytomegalovirus
  siRNA target sequence
ccatccagtaccgcaacaa

SEQ ID NO: 33 - DNA, 19 bp, Cytomegalovirus
  siRNA target sequence
gtaccgcaacaagattatc

SEQ ID NO: 34 - DNA, 19 bp, Cytomegalovirus
  siRNA target sequence
ccgcaacaagattatcgat

SEQ ID NO: 35 - DNA, 19 bp, Cytomegalovirus
  siRNA target sequence
agaagagcaaacgcatctc

SEQ ID NO: 36 - DNA, 19 bp, Cytomegalovirus
  siRNA target sequence
aacgcatctccgagttgga

SEQ ID NO: 37 - DNA, 19 bp, Cytomegalovirus
  siRNA target sequence
caacgagaaggtgcgcaat

SEQ ID NO: 38 - DNA, 19 bp, Cytomegalovirus
  siRNA target sequence
caccaatcgctctcttgag

SEQ ID NO: 39 - DNA, 19 bp, Cytomegalovirus
  siRNA target sequence
ccaatcgctctcttgagta

SEQ ID NO: 40 - DNA, 19 bp, Cytomegalovirus
  siRNA target sequence
atcgctctcttgagtacaa

SEQ ID NO: 41 - DNA, 19 bp, Cytomegalovirus
  siRNA target sequence
ccatgcaggtgaacaacaa

SEQ ID NO: 42 - DNA, 19 bp, Cytomegalovirus
  siRNA target sequence
cagccgatgcttgtaacga

SEQ ID NO: 43 - DNA, 19 bp, Cytomegalovirus
  siRNA target sequence
ttaccgcaacatgatcatc

SEQ ID NO: 44 - DNA, 19 bp, Cytomegalovirus
  siRNA target sequence
tgttctatcgagagattaa

SEQ ID NO: 45 - DNA, 19 bp, Cytomegalovirus
  siRNA target sequence
cagaacacggctacagtat

SEQ ID NO: 46 - DNA, 19 bp, Cytomegalovirus
  siRNA target sequence
gaacacggctacagtatct

SEQ ID NO: 47 - DNA, 19 bp, Cytomegalovirus
  siRNA target sequence
cttgtgatatcgaggtaga

SEQ ID NO: 48 - DNA, 19 bp, Cytomegalovirus
  siRNA target sequence
tcgaggtagactgcgatgt

SEQ ID NO: 49 - DNA, 19 bp, Cytomegalovirus
  siRNA target sequence
tgcctgtccttcgatatcg

SEQ ID NO: 50 - DNA, 19 bp, Cytomegalovirus
  siRNA target sequence
acactatggccgagcttta

SEQ ID NO: 51 - DNA, 19 bp, Cytomegalovirus
  siRNA target sequence
cactatggccgagctttac

SEQ ID NO: 52 - DNA, 19 bp, Cytomegalovirus
  siRNA target sequence
ttggtgcgcgatctgttca

SEQ ID NO: 53 - DNA, 19 bp, Cytomegalovirus
  siRNA target sequence
acgaatagcgttgctgtgt

SEQ ID NO: 54 - DNA, 19 bp, Cytomegalovirus
  siRNA target sequence
cctaacgctgctatcatct

SEQ ID NO: 55 - DNA, 19 bp, Cytomegalovirus
  siRNA target sequence
atgcatgcgcgagtgtcaa

SEQ ID NO: 56 - DNA, 19 bp, Cytomegalovirus
  siRNA target sequence
acagatggcgctcaaagta

SEQ ID NO: 57 - DNA, 19 bp, Cytomegalovirus
  siRNA target sequence
aagtaacgtgcaacgcttt

SEQ ID NO: 58 - DNA, 19 bp, Cytomegalovirus
  siRNA target sequence
agtaacgtgcaacgctttc

SEQ ID NO: 59 - DNA, 19 bp, Cytomegalovirus
  siRNA target sequence
gtaacgtgcaacgctttct

SEQ ID NO: 60 - DNA, 19 bp, Cytomegalovirus
  siRNA target sequence
aaaggtcttcgtctctctt

SEQ ID NO: 61 - DNA, 19 bp, Cytomegalovirus
  siRNA target sequence
aaggtcttcgtctctctta

SEQ ID NO: 62 - DNA, 19 bp, Cytomegalovirus
  siRNA target sequence
tgatctgcaagaaacgtta

SEQ ID NO: 63 - DNA, 19 bp, Cytomegalovirus
  siRNA target sequence
tctgcaagaaacgttacat

SEQ ID NO: 64 - DNA, 19 bp, Cytomegalovirus
  siRNA target sequence
aacgttacatcggcaaagt

SEQ ID NO: 65 - DNA, 19 bp, Cytomegalovirus
  siRNA target sequence
acgttacatcggcaaagtg

SEQ ID NO: 66 - DNA, 19 bp, Cytomegalovirus
  siRNA target sequence
catctcgctgtaccgtcaa

SEQ ID NO: 67 - DNA, 19 bp, Cytomegalovirus
  siRNA target sequence
tctcgctgtaccgtcaatc

SEQ ID NO: 68 - DNA, 19 bp, Cytomegalovirus
  siRNA target sequence
ttgccgtcattaagcgatt

SEQ ID NO: 69 - DNA, 19 bp, Cytomegalovirus
  siRNA target sequence
cgccgacaagtactttgag

SEQ ID NO: 70 - DNA, 19 bp, Cytomegalovirus
  siRNA target sequence
tttgttatgccgtggacat

SEQ ID NO: 71 - DNA, 19 bp, Cytomegalovirus
  siRNA target sequence
caacgtcacggtacatcga

SEQ ID NO: 72 - DNA, 19 bp, Cytomegalovirus
  siRNA target sequence
cggtacatcgacgtttcca

SEQ ID NO: 73 - DNA, 19 bp, Cytomegalovirus
  siRNA target sequence
atcaccagtgtcgtgtatg

SEQ ID NO: 74 - DNA, 19 bp, Cytomegalovirus
  siRNA target sequence
tcaccagtgtcgtgtatgc

SEQ ID NO: 75 - DNA, 19 bp, Cytomegalovirus
  siRNA target sequence
gtgtcgtgtatgccacttt

SEQ ID NO: 76 - DNA, 19 bp, Cytomegalovirus
  siRNA target sequence
tgccactttgacattacac

SEQ ID NO: 77 - DNA, 19 bp, Cytomegalovirus
  siRNA target sequence
cggaggcgttgctctttaa

SEQ ID NO: 78 - DNA, 19 bp, Cytomegalovirus
  siRNA target sequence
aaagtccgagctggtttcg

SEQ ID NO: 79 - DNA, 19 bp, Cytomegalovirus
  siRNA target sequence
tacgtcaaggcgagcgttt

SEQ ID NO: 80 - DNA, 19 bp, Cytomegalovirus
  siRNA target sequence
acaaacgccgtaaggaaac

SEQ ID NO: 81 - DNA, 19 bp, Cytomegalovirus
  siRNA target sequence
caaacgccgtaaggaaacc

SEQ ID NO: 82 - DNA, 19 bp, Cytomegalovirus
  siRNA target sequence
gcagcagcaacaacgttac

SEQ ID NO: 83 - DNA, 19 bp, Cytomegalovirus
  siRNA target sequence
gcaacaacgttacgatgaa

SEQ ID NO: 84 - DNA, 19 bp, Cytomegalovirus
  siRNA target sequence
gagttctacgttactttcg

SEQ ID NO: 85 - DNA, 19 bp, Cytomegalovirus
  siRNA target sequence
ctactactaccgtgtgtac

SEQ ID NO: 86 - DNA, 19 bp, Cytomegalovirus
  siRNA target sequence
gacatggtagatctgaatc

SEQ ID NO: 87 - DNA, 19 bp, Cytomegalovirus
  siRNA target sequence
gtctgcgttaacctgtgta

SEQ ID NO: 88 - DNA, 19 bp, Cytomegalovirus
  siRNA target sequence
agccatacttctcgtacga

SEQ ID NO: 89 - DNA, 19 bp, Cytomegalovirus
  siRNA target sequence
tagagccaacgagactatc

SEQ ID NO: 90 - DNA, 19 bp, Cytomegalovirus
  siRNA target sequence
gagccaacgagactatcta

SEQ ID NO: 91 - DNA, 19 bp, Cytomegalovirus
  siRNA target sequence
gccaacgagactatctaca

SEQ ID NO: 92 - DNA, 19 bp, Cytomegalovirus
  siRNA target sequence
acgagactatctacaacac

SEQ ID NO: 93 - DNA, 19 bp, Cytomegalovirus
  siRNA target sequence
cgagactatctacaacact

SEQ ID NO: 94 - DNA, 19 bp, Cytomegalovirus
  siRNA target sequence
cggatcttattcgctttga

SEQ ID NO: 95 - DNA, 19 bp, Cytomegalovirus
  siRNA target sequence
tcttattcgctttgaacgt

SEQ ID NO: 96 - DNA, 19 bp, Cytomegalovirus
  siRNA target sequence
ttcgctttgaacgtaatat

SEQ ID NO: 97 - DNA, 19 bp, Cytomegalovirus
  siRNA target sequence
cctcgatgaagcctatcaa

SEQ ID NO: 98 - DNA, 19 bp, Cytomegalovirus
  siRNA target sequence
tgaagcctatcaatgaaga

SEQ ID NO: 99 - DNA, 19 bp, Cytomegalovirus
  siRNA target sequence
tcaacaagtttgctcaatg

SEQ ID NO: 100 - DNA, 19 bp, Cytomegalovirus
  siRNA target sequence
gttcctacagccgcgttat

SEQ ID NO: 101 - DNA, 19 bp, Cytomegalovirus
  siRNA target sequence
tcgtgagacctgtaatctg

SEQ ID NO: 102 - DNA, 19 bp, Cytomegalovirus
  siRNA target sequence
actgtatgctgaccatcac

SEQ ID NO: 103 - DNA, 19 bp, Cytomegalovirus
  siRNA target sequence
ctgtatgctgaccatcact

SEQ ID NO: 104 - DNA, 19 bp, Cytomegalovirus
  siRNA target sequence
acggaaccaatcgcaatgc

SEQ ID NO: 105 - DNA, 19 bp, Cytomegalovirus
  siRNA target sequence
agcctcggaacgtactatc

SEQ ID NO: 106 - DNA, 19 bp, Cytomegalovirus
  siRNA target sequence
cgtgatgaggctataaata

SEQ ID NO: 107 - DNA, 19 bp, Cytomegalovirus
  siRNA target sequence
aacgtgtccgtcttcgaaa

SEQ ID NO: 108 - DNA, 19 bp, Cytomegalovirus
  siRNA target sequence
acgtgtccgtcttcgaaac

SEQ ID NO: 109 - DNA, 19 bp, Cytomegalovirus
  siRNA target sequence
cgtttggccaatcgatcca

SEQ ID NO: 110 - DNA, 19 bp, Cytomegalovirus
  siRNA target sequence
atcgatccagtctgaatat

SEQ ID NO: 111 - DNA, 19 bp, Cytomegalovirus
  siRNA target sequence
tcgatccagtctgaatatc

SEQ ID NO: 112 - DNA, 19 bp, Cytomegalovirus
  siRNA target sequence
gaagtacgagtgacaataa

SEQ ID NO: 113 - DNA, 19 bp, Cytomegalovirus
  siRNA target sequence
gtacgagtgacaataatac

SEQ ID NO: 114 - DNA, 19 bp, Cytomegalovirus
  siRNA target sequence
gcatggaatcggtgcacaa

SEQ ID NO: 115 - DNA, 19 bp, Cytomegalovirus
  siRNA target sequence
tggaatcggtgcacaatct

SEQ ID NO: 116 - DNA, 19 bp, Cytomegalovirus
  siRNA target sequence
cgttgcgcggttacatcaa

SEQ ID NO: 117 - DNA, 19 bp, Cytomegalovirus
  siRNA target sequence
tttacaacaaaccgattgc

SEQ ID NO: 118 - DNA, 19 bp, Cytomegalovirus
  siRNA target sequence
ggtgctgcgtgatatgaac

SEQ ID NO: 119 - DNA, 19 bp, Cytomegalovirus
  siRNA target sequence
atttcgccaacagctcgta

SEQ ID NO: 120 - DNA, 19 bp, Cytomegalovirus
  siRNA target sequence
acagctcgtacgtgcagta

SEQ ID NO: 121 - DNA, 19 bp, Cytomegalovirus
  siRNA target sequence
gtacgtggactacctcttc

SEQ ID NO: 122 - DNA, 19 bp, Cytomegalovirus
  siRNA target sequence
cgtggactacctcttcaaa

SEQ ID NO: 123 - DNA, 19 bp, Cytomegalovirus
  siRNA target sequence
agagatcatgcgcgaattc

SEQ ID NO: 124 - DNA, 19 bp, Cytomegalovirus
  siRNA target sequence
gagatcatgcgcgaattca

SEQ ID NO: 125 - DNA, 19 bp, Cytomegalovirus
  siRNA target sequence
gatcatgcgcgaattcaac

SEQ ID NO: 126 - DNA, 19 bp, Cytomegalovirus
  siRNA target sequence
tcatgcgcgaattcaactc

SEQ ID NO: 127 - DNA, 19 bp, Cytomegalovirus
  siRNA target sequence
tgcgcgaattcaactcgta

SEQ ID NO: 128 - DNA, 19 bp, Cytomegalovirus
  siRNA target sequence
agtacgtggaggacaaggt

SEQ ID NO: 129 - DNA, 19 bp, Cytomegalovirus
  siRNA target sequence
gtacgtggaggacaaggta

SEQ ID NO: 130 - DNA, 19 bp, Cytomegalovirus
  siRNA target sequence
tagccgtagtcattatcac

SEQ ID NO: 131 - DNA, 19 bp, Cytomegalovirus
  siRNA target sequence
gccgtagtcattatcactt

SEQ ID NO: 132 - DNA, 19 bp, Cytomegalovirus
  siRNA target sequence
ccaaagacacgtcgttaca

SEQ ID NO: 133 - DNA, 19 bp, Cytomegalovirus
  siRNA target sequence
gaacggtacagattctttg

SEQ ID NO: 134 - DNA, 19 bp, Cytomegalovirus
  siRNA target sequence
aacggctacagacacttga

SEQ ID NO: 135 - DNA, 19 bp, Cytomegalovirus
  siRNA target sequence
cttgaaagactccgacgaa

SEQ ID NO: 136 - DNA, 19 bp, Cytomegalovirus
  siRNA target sequence
ctccgacgaagaagagaac

SEQ ID NO: 137 - DNA, 19 bp, Cytomegalovirus
  siRNA target sequence
cctaccttcgcaacgatat

SEQ ID NO: 138 - DNA, 19 bp, Cytomegalovirus
  siRNA target sequence
cgcatttcacctactactc

SEQ ID NO: 139 - DNA, 19 bp, Cytomegalovirus
  siRNA target sequence
ttccatatgcctcgatgtc

SEQ ID NO: 140 - DNA, 19 bp, Cytomegalovirus
  siRNA target sequence
ggtagatctgaccgaaacc

SEQ ID NO: 141 - DNA, 19 bp, Cytomegalovirus
  siRNA target sequence
cttaacacctacgcattgg

SEQ ID NO: 142 - DNA, 19 bp, Cytomegalovirus
  siRNA target sequence
acacctacgcattggtatc

SEQ ID NO: 143 - DNA, 19 bp, Cytomegalovirus
  siRNA target sequence
ctacatcggccacacttta

SEQ ID NO: 144 - DNA, 19 bp, Cytomegalovirus
  siRNA target sequence
cctcatggacgaactacgt

SEQ ID NO: 145 - DNA, 19 bp, Cytomegalovirus
  siRNA target sequence
tcaacgcgacaactttata

SEQ ID NO: 146 - DNA, 19 bp, Cytomegalovirus
  siRNA target sequence
caactttatactacgacaa

SEQ ID NO: 147 - DNA, 19 bp, Cytomegalovirus
  siRNA target sequence
actttatactacgacaaac

SEQ ID NO: 148 - DNA, 19 bp, Cytomegalovirus
  siRNA target sequence
gctcctggtactagttaag

SEQ ID NO: 149 - DNA, 19 bp, Cytomegalovirus
  siRNA target sequence
ctagttaagaaagctcaac

SEQ ID NO: 150 - DNA, 19 bp, Cytomegalovirus
  siRNA target sequence
gctcaactaaaccgtcact

SEQ ID NO: 151 - DNA, 19 bp, Cytomegalovirus
  siRNA target sequence
aaccgtcactcctatctca

SEQ ID NO: 152 - DNA, 19 bp, Cytomegalovirus
  siRNA target sequence
ccgtcactcctatctcaaa

SEQ ID NO: 153 - DNA, 19 bp, Cytomegalovirus
  siRNA target sequence
cgctgtagacgtactcaaa

SEQ ID NO: 154 - DNA, 19 bp, Cytomegalovirus
  siRNA target sequence
agcggtcgatgtcaaatgt

SEQ ID NO: 155 - DNA, 19 bp, Cytomegalovirus
  siRNA target sequence
gcggtcgatgtcaaatgtt

SEQ ID NO: 156 - DNA, 19 bp, Cytomegalovirus
  siRNA target sequence
ggccgcactcttacaaata

SEQ ID NO: 157 - DNA, 19 bp, Cytomegalovirus
  siRNA target sequence
tgatcacctgcctctcaca

SEQ ID NO: 158 - DNA, 19 bp, Cytomegalovirus
  siRNA target sequence
gagacgcgaaatcttcatc

SEQ ID NO: 159 - DNA, 19 bp, Cytomegalovirus
  siRNA target sequence
gacgcgaaatcttcatcgt

SEQ ID NO: 160 - DNA, 19 bp, Cytomegalovirus
  siRNA target sequence
ttggccgagctatcacact

SEQ ID NO: 161 - DNA, 19 bp, Cytomegalovirus
  siRNA target sequence
ctttacgcagttgctagct

SEQ ID NO: 162 - DNA, 19 bp, Cytomegalovirus
  siRNA target sequence
atacctcagcgacctgtac

SEQ ID NO: 163 - DNA, 19 bp, Cytomegalovirus
  siRNA target sequence
tacctcagcgacctgtaca

SEQ ID NO: 164 - DNA, 19 bp, Cytomegalovirus
  siRNA target sequence
acacgtcagttatgtcgta

SEQ ID NO: 165 - DNA, 19 bp, Cytomegalovirus
  siRNA target sequence
cacgtcagttatgtcgtaa

SEQ ID NO: 166 - DNA, 19 bp, Cytomegalovirus
  siRNA target sequence
aacggacagtcaaactaaa

SEQ ID NO: 167 - DNA, 19 bp, Cytomegalovirus
  siRNA target sequence
acggacagtcaaactaaat

SEQ ID NO: 168 - DNA, 19 bp, Cytomegalovirus
  siRNA target sequence
cggacagtcaaactaaatg

SEQ ID NO: 169 - DNA, 19 bp, Cytomegalovirus
  siRNA target sequence
cgcaaggcgtcatcaacat

SEQ ID NO: 170 - DNA, 19 bp, Cytomegalovirus
  siRNA target sequence
caacgaagtggtggtctca

SEQ ID NO: 171 - DNA, 19 bp, Cytomegalovirus
  siRNA target sequence
aaacggtacggtcctagaa

SEQ ID NO: 172 - DNA, 19 bp, Cytomegalovirus
  siRNA target sequence
aacggtacggtcctagaag

SEQ ID NO: 173 - DNA, 19 bp, Cytomegalovirus
  siRNA target sequence
acggtacggtcctagaagt

SEQ ID NO: 174 - DNA, 19 bp, Cytomegalovirus
  siRNA target sequence
cggtacggtcctagaagta

SEQ ID NO: 175 - DNA, 19 bp, Cytomegalovirus
  siRNA target sequence
cagtcgtctcctcatgatg

SEQ ID NO: 176 - DNA, 19 bp, Cytomegalovirus
  siRNA target sequence
gtcgtctcctcatgatgtc

SEQ ID NO: 177 - DNA, 1476 bp, Cytomegalovirus
  viral gene sequence
atggagtcctctgccaagagaaagatggaccctgataatcctgacgagggcccttcctccaaggtgccacggcccgagacacccgtgaccaaggccacgacgttcctgcagactatgttgaggaaggaggttaacagtcagctgagtctgggagacccgctgtttccagagttggccgaagaatccctcaaaacttttgaacaagtgaccgaggattgcaacgagaaccccgagaaagatgtcctggcagaactcgtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgaccagtaa

SEQ ID NO: 178 - DNA, 1743 bp, Cytomegalovirus
  viral gene sequence
atggagtcctctgccaagagaaagatggaccctgataatcctgacgagggcccttcctccaaggtgccacggcccgagacacccgtgaccaaggccacgacgttcctgcagactatgttgaggaaggaggttaacagtcagctgagtctgggagacccgctgtttccagagttggccgaagaatccctcaaaacttttgaacaagtgaccgaggattgcaacgagaaccccgagaaagatgtcctggcagaactcggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacacatccacccgaagtggcgcagcgcacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcagtaa

SEQ ID NO: 179 - DNA, 3729 bp, Cytomegalovirus
  viral gene sequence
atgtttttcaacccgtatctgagcggcggcgtgaccggcggtgcggtcgcgggtggccggcgtcagcgttcgcagcccggctccgcgcagggctcgggcaagcggccgccacagaaacagtttttgcagatcgtgccgcgaggtgtcatgttcgacggtcagacggggttgatcaagcataagacgggacggctgcctctcatgttctatcgagagattaaacatttgttgagtcatgacatggtttggccgtgtccttggcgcgagaccctggtgggtcgcgtggtgggacctattcgttttcacacctacgatcagacggacgccgtgctcttcttcgactcgcccgaaaacgtgtcgccgcgctatcgtcagcatctggtgccttcggggaacgtgttgcgtttcttcggggccacagaacacggctacagtatctgcgtcaacgttttcgggcagcgcagctacttttactgtgagtacagcgacaccgataggctgcgtgaggtcattgccagcgtgggcgaactagtgcccgaaccgcggacgccatacgccgtgtctgtcacgccggccaccaagacctccatctatgggtacgggacgcgacccgtgcccgatttgcagtgtgtgtctatcagcaactggaccatggccagaaaaatcggcgagtatctgctggagcagggttttcccgtgtacgaggtccgtgtggatccgctgacgcgtttggtcatcgatcggcggatcaccacgttcggctggtgctccgtgaatcgttacgactggcggcagcagggtcgcgcgtcgacttgtgatatcgaggtagactgcgatgtctctgacctggtggctgtgcccgacgacagctcgtggccgcgctatcgatgcctgtccttcgatatcgagtgcatgagcggcgagggtggttttccctgcgccgagaagtccgatgacattgtcattcagatctcgtgcgtgtgctacgagacggggggaaacaccgccgtggatcaggggatcccaaacgggaacgatggtcggggctgcacttcggagggtgtgatctttgggcactcgggtcttcatctctttacgatcggcacctgcgggcaggtgggcccagacgtggacgtctacgagttcccttccgaatacgagctgctgctgggctttatgcttttctttcaacggtacgcgccggcctttgtgaccggttacaacatcaactcttttgacttgaagtacatcctcacgcgtctcgagtacctgtataaggtggactcgcagcgcttctgcaagttgcctacggcgcagggcggccgtttctttttacacagccccgccgtgggttttaagcggcagtacgccgccgcttttccctcggcttctcacaacaatccggccagcacggccgccaccaaggtgtatattgcgggttcggtggttatcgacatgtaccctgtatgcatggccaagactaactcgcccaactataagctcaacactatggccgagctttacctgcggcaacgcaaggatgacctgtcttacaaggacatcccgcgttgtttcgtggctaatgccgagggccgcgcccaggtaggccgttactgtctgcaggacgccgtattggtgcgcgatctgttcaacaccattaattttcactacgaggccggggccatcgcgcggctggctaaaattccgttgcggcgtgtcatctttgacggacagcagatccgtatctacacctcgctgctggacgagtgcgcctgccgcgattttatcctgcccaaccactacagcaaaggtacgacggtgcccgaaacgaatagcgttgctgtgtcacctaacgctgctatcatctctaccgccgctgtgcccggcgacgcgggttctgtggcggctatgtttcagatgtcgccgcccttgcaatctgcgccgtccagtcaggacggcgtttcacccggctccggcagtaacagtagtagcagcgtcggcgttttcagcgtcggctccggcagtagtggcggcgtcggcgtttccaacgacaatcacggcgccggcggtactgcggcggtttcgtaccagggcgccacggtgtttgagcccgaggtgggttactacaacgaccccgtggccgtgttcgactttgccagcctctacccttccatcatcatggcccacaacctctgctactccaccctgctggtgccgggtggcgagtaccctgtggaccccgccgacgtatacagcgtcacgctagagaacggcgtgacccaccgctttgtgcgtgcttcggtgcgcgtctcggtgctctcggaactgctcaacaagtgggtttcgcagcggcgtgccgtgcgcgaatgcatgcgcgagtgtcaagaccctgtgcgccgtatgctgctcgacaaggaacagatggcgctcaaagtaacgtgcaacgctttctacggttttaccggcgtggtcaacggtatgatgccgtgtctgcccatcgccgccagcatcacgcgcatcggtcgcgacatgctagagcgcacggcgcggttcatcaaagacaacttttcagagccgtgttttttgcacaatttttttaatcaggaagactatgtagtgggaacgcgggagggggattcggaggagagcagcgcgttaccggaggggctcgaaacatcgtcagggggctcgaacgaacggcgggtggaggcgcgggtcatctacggggacacggacagcgtgtttgtccgctttcgtggcctgacgccgcaggctctggtggcgcgtgggcccagcctggcgcactacgtgacggcctgtctttttgtggagcccgtcaagctggagtttgaaaaggtcttcgtctctcttatgatgatctgcaagaaacgttacatcggcaaagtggagggcgcctcgggtctgagcatgaagggcgtggatctggtgcgcaagacggcctgcgagttcgtcaagggcgtcacgcgtgacgtcctctcgctgctctttgaggatcgcgaggtctcggaagcagccgtgcgcctgtcgcgcctctcactcgatgaagtcaagaagtacggcgtgccacgcggtttctggcgtatcttacgccgcttggtgcaggcccgcgacgatctgtacctgcaccgtgtgcgtgtcgaggacctggtgctttcgtcggtgctctctaaggacatctcgctgtaccgtcaatctaacctgccgcacattgccgtcattaagcgattggcggcccgttctgaggagctaccctcggtcggggatcgggtcttttacgttctgacggcgcccggtgtccggacggcgccgcagggttcctccgacaacggtgattctgtaaccgccggcgtggtttcccggtcggacgcgattgatggcacggacgacgacgctgacggcggcggggtagaggagagcaacaggagaggaggagagccggcaaagaagagggcgcggaaaccaccgtcggccgtgtgcaactacgaggtagccgaagatccgagctacgtgcgcgagcacggcgtgcccattcacgccgacaagtactttgagcaggttctcaaggctgtaactaacgtgctgtcgcccgtctttcccggcggcgaaaccgcgcgcaaggacaagtttttgcacatggtgctgccgcggcgcttgcacttggagccggcttttctgccgtacagtgtcaaggcgcacgaatgctgttga

SEQ ID NO: 180 - DNA, 2124 bp, Cytomegalovirus
  viral gene sequence
atgtcctccgcacttcggtctcgggctcgctcggcctcgctcggaacgacgactcagggctgggatccgccgccattgcgtcgtcccagcagggcgcgccggcgccagtggatgcgcgaagctgcgcaggccgccgctcaagccgcggtgcaggccgcgcaggccgccgccgctcaggtcgcccaggctcacgttgatgaaaacgaggtcgtggatctgatggccgacgaggccggcggcggcgtcaccactttgaccaccctgagttccgtcagcacaaccaccgtgcttggacacgcgactttttccgcatgcgttcgaagtgacgtgatgcgtgacggagaaaaagaggacgcggcttcggacaaggagaacctgcgtcggcccgtagtgccgtccacgtcgtctcgcggcagcgccgccagcggcgacggttaccacggcttgcgctgccgcgaaacttcggccatgtggtcgttcgagtacgatcgcgacggcgacgtgaccagcgtacgccgcgctctcttcaccggcggcagcgacccctcggacagcgtgagcggcgtccgcggtggacgcaaacgcccgttgcgtccgccgttggtgtcgctggcccgcaccccgctgtgccgacgtcgtgtgggcggtgtggacgcggtgctcgaagaaaacgacgtggagctgcgcgcggaaagtcaggacagcgccgtggcatcgggcccgggccgcattccgcagccgctcagcggtagttccggggaggaatccgccacggcggtggaggccgactccacgtcacacgacgacgtgcattgcacctgttccaacgaccagatcatcaccacgtccatccgcggccttacgtgcgacccgcgtatgttcttgcgccttacgcatcccgagctctgcgagctctctatctcctacctgctggtctacgtgcccaaagaggacgatttttgccacaagatttgttatgccgtggacatgagcgacgagagctaccgcctgggccagggctccttcggcgaggtctggccgctcgatcgctatcgcgtggtcaaggtggcgcgtaagcacagcgagacggtgctcacggtctggatgtcgggcctgatccgcacgcgcgccgctggcgagcaacagcagccgccgtcgctggtgggcacgggcgtgcaccgcggtctgctcacggccacgggctgctgtctgctgcacaacgtcacggtacatcgacgtttccacacagacatgtttcatcacgaccagtggaagctggcgtgcatcgacagctaccgacgtgccttttgcacgttggccgacgctatcaaatttctcaatcaccagtgtcgtgtatgccactttgacattacacccatgaacgtgctcatcgacgtgaacccgcacaaccccagcgagatcgtgcgcgccgcgctgtgcgattacagcctcagcgagccctatccggattacaacgagcgctgtgtggccgtctttcaggagacgggtacggcgcgccgcatccccaactgctcgcaccgtctgcgcgaatgttaccaccctgctttccgacccatgccgctgcagaagctgctcatctgcgacccgcacgcgcgtttccccgtagccggcctacggcgttattgcatgtcggagctgtcggcgctgggtaacgtgctgggcttttgcctcatgcggctgttggaccggcgcggtctggacgaggtgcgcatgggcacggaggcgttgctctttaagcacgccggcgcggcctgccgcgcgttggagaacggtaagctcacgcactgctccgacgcctgtctgctcattctggcggcgcaaatgagctacggcgcctgtctcctgggcgagcatggcgccgcgctggtgtcgcacacgctgcgctttgtggaggccaagatgtcctcgtgtcgcgtacgcgcctttcgccgcttctaccacgaatgctcgcagaccatgctgcacgaatacgtcagaaagaacgtggagcgtctgttggccacgagcgacgggctgtatttatataacgcctttcggcgcaccaccagcataatctgcgaggaggaccttgacggtgactgccgccaactgttccccgagtaa

SEQ ID NO: 181 - DNA, 2127 bp, Cytomegalovirus
  viral gene sequence
atgacgatggacgagcagcagtcgcaggctgtggcgccggtctacgtgggcggctttctcgcccgctacgaccagtctccggacgaggccgaattgctgttgccgcgggacgtagtggagcactggttgcacgcgcagggccagggacagccttcgttgtcggtcgcgctcccgctcaacatcaaccacgacgacacggccgttgtaggacacgttgcggcgatgcagagcgtccgcgacggtcttttttgcctgggctgcgtcacttcgcccaggtttctggagattgtacgccgcgcttcggaaaagtccgagctggtttcgcgcgggcccgtcagtccgctgcagccagacaaggtggtggagtttctcagcggcagctacgccggcctctcgctctccagccggcgctgcgacgacgtggaggccgcgacgtcgctttcgggctcggaaaccacgccgttcaaacacgtggctttgtgcagcgtgggtcggcgtcgcggtacgttggccgtgtacgggcgcgatcccgagtgggtcacacagcggtttccagacctcacggcggccgaccgtgacgggctacgtgcacagtggcagcgctgcggcagcactgctgtcgacgcgtcgggcgatccctttcgctcagacagctacggcctgttgggcaacagcgtggacgcgctctacatccgtgagcgactgcccaagctgcgctacgacaagcaactagtcggcgtgacggagcgcgagtcatacgtcaaggcgagcgtttcgcctgaggcggcgtgcgatattaaagcggcgtccgccgagcgttcgggcgacagccgcagtcaggccgccacgccggcggctggggcgcgcgttccctcttcgtccccgtcgcctccagtcgaaccgccatctcctgtacagccgcctgcgcttccagcgtcgccgtccgttcttcccgcggaatcaccgccgtcgctttctccctcggagccggcagaggcggcgtccatgtcgcaccctctgagtgctgcggttcccgccgctacggctcctccaggtgctaccgtggcaggtgcgtcgccggctgtgtcgtctctagcgtggcctcacgacggagtttatttacccaaagacgcttttttctcgctacttggggccagtcgctcggcagtgcccgtcatgtatcccggcgccgtagcggcccctccttctgcttcgccagcaccgctgcctttgccgtcttatcccgcgtcctacggcgcccccgtcgtgggttacgaccagttggcggcacgtcactttgcggactacgtggatccccattatcccgggtggggtcggcgttacgagcccgcgccgtctttgcatccgtcttatcccgtgccgccgccaccatcaccggcctattaccgtcggcgcgactctccgggcggtatggatgaaccaccgtccggatgggagcgttacgacggtggtcaccgtggtcagtcgcagaagcagcaccgtcacgggggcagcggcggacacaacaaacgccgtaaggaaaccgcggcggcgtcgtcgtcgtcctcggacgaagacttgagtttcccaggcgaggccgagcacggccgggcacgaaagcgtctaaaaagtcacgtcaatagcgacggtggaagtggcgggcacgcgggttccaatcagcagcagcaacaacgttacgatgaactgcgggatgccattcacgagctgaaacgcgatctgtttgctgcgcggcagagttctacgttactttcggcggctcttccctctgcggcctcttcctccccaactactactaccgtgtgtactcccaccggcgagctgacgagtggcggaggagaaacacccacggcacttctatccggaggtgccaaggtagctgagcgcgctcaggccggcgtggtgaacgccagttgccgcctcgctaccgcgtcgggttctgaggcggcaacggccgggccctcgacggcaggttcttcttcctgcccggctagtgtcgtgttagccgccgctgctgcccaagccgccgcagcttcccagagcccgcccaaagacatggtagatctgaatcggcggatttttgtggctgcgctcaataagctcgagtaa

SEQ ID NO: 182 - DNA, 2721 bp, Cytomegalovirus
  viral gene sequence
atggaatccaggatctggtgcctggtagtctgcgttaacctgtgtatcgtctgtctgggtgctgcggtttcctcttctagtacttcccatgcaacttcttctactcacaatggaagccatacttctcgtacgacgtctgctcaaacccggtcagtctattctcaacacgtaacgtcttctgaagccgtcagtcatagagccaacgagactatctacaacactaccctcaagtacggagatgtggtgggagtcaacactaccaagtacccctatcgcgtgtgttctatggcccagggtacggatcttattcgctttgaacgtaatatcatctgcacctcgatgaagcctatcaatgaagacttggatgagggcatcatggtggtctacaagcgcaacatcgtggcgcacacctttaaggtacgggtctaccaaaaggttttgacgtttcgtcgtagctacgcttacatctacaccacttatctgctgggcagcaatacggaatacgtggcgcctcctatgtgggagattcatcacatcaacaagtttgctcaatgctacagttcctacagccgcgttataggaggcacggttttcgtggcatatcatagggacagttatgaaaacaaaaccatgcaattaattcccgacgattattccaacacccacagtacccgttacgtgacggtcaaggatcagtggcacagccgcggcagcacctggctctatcgtgagacctgtaatctgaactgtatgctgaccatcactactgcgcgctccaagtatccttatcatttttttgcaacttccacgggtgatgtggtttacatttctcctttctacaacggaaccaatcgcaatgccagctactttggagaaaacgccgacaagtttttcattttcccgaactacaccatcgtttccgactttggaagacccaacgctgcgccagaaacccataggttggtggcttttctcgaacgtgccgactcggtgatctcttgggatatacaggacgagaagaatgtcacctgccagctcaccttctgggaagcctcggaacgtactatccgttccgaagccgaagactcgtaccacttttcttctgccaaaatgactgcaacttttctgtctaagaaacaagaagtgaacatgtccgactccgcgctggactgcgtacgtgatgaggctataaataagttacagcagattttcaatacttcatacaatcaaacatatgaaaaatacggaaacgtgtccgtcttcgaaaccagcggcggtctggtggtgttctggcaaggcatcaagcaaaaatctttggtggaattggaacgtttggccaatcgatccagtctgaatatcactcataggaccagaagaagtacgagtgacaataatacaactcatttgtccagcatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggaatcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaagtacgtggaggacaaggtagtcgacccgctaccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagccttcaccatcatcctcgtggccatagccgtagtcattatcacttatttgatctatactcgacagcggcgtctgtgcacgcagccgctgcagaacctctttccctatctggtgtccgccgacgggaccaccgtgacgtcgggcagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcagaacggtacagattctttggacggacagactggcacgcaggacaagggacagaagcctaacctgctagaccggctgcgacatcgcaaaaacggctacagacacttgaaagactccgacgaagaagagaacgtctga

SEQ ID NO: 183 - DNA, 2232 bp, Cytomegalovirus
  viral gene sequence
atgcggcccggcctccccccctacctcactgtcttcaccgtctacctcctcagtcacctaccttcgcaacgatatggcgcggacgccgcatccgaagcgctggaccctcacgcatttcacctactactcaacacctacgggagacccatccgcttcctgcgtgaaaacaccacccagtgcacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctagaaagataccaacagagacttaacacctacgcattggtatccaaagacctggccagctaccgatctttttcgcagcagctgaaggcacaagacagcctgggtcagcagcccaccaccgtgccaccgcccattgatctgtcaatacctcacgtttggatgccaccccaaaccactccacacgactggaagggatcgcacaccacctcgggactacatcggccacactttaaccagacctgtatcctctttgatggacacgatctgcttttcagcaccgttacgccctgtctgcaccagggcttttacctcatggacgaactacgttacgttaaaatcacactgaccgaggacttcttcgtagttacggtatctatagacgacgacacacccatgctgcttatcttcggtcatcttccacgcgtactcttcaaagcgccctatcaacgcgacaactttatactacgacaaactgaaaaacacgagctcctggtactagttaagaaagctcaactaaaccgtcactcctatctcaaagactcggactttctcgacgccgcactcgacttcaactacctggacctcagcgcactgttacgtaacagctttcaccgttacgctgtagacgtactcaaaagcggtcgatgtcaaatgttggaccgccgcacggtagaaatggccttcgcctacgcattagcactgttcgcggcagcccgacaagaagaggccggcaccgaaatctccatcccacgagccctagaccgccaggccgcactcttacaaatacaagaatttatgatcacctgcctctcacaaacaccaccacgcaccacattgctgctatatcccacagccgtggacctggccaaacgagccctctggacgccggaccagatcaccgacatcaccagcctcgtacgcctggtctacatactttctaaacagaatcagcaacatctcattccccagtgggcactacgacagatcgccgactttgccctacaattacacaaaacgcacctggcctcttttctttcagccttcgcgcgccaagaactctacctcatgggcagcctcgtccactccatgttggtacatacgacggagagacgcgaaatcttcatcgtagaaacgggcctctgttcattggccgagctatcacactttacgcagttgctagctcatccgcaccacgaatacctcagcgacctgtacacaccctgttccagtagcgggcgacgcgatcactcgctcgaacgcctcacgcgtctcttccccgatgccaccgttcctgctaccgttcccgccgccctctccatcctatctaccatgcaaccaagcacgctggaaaccttccccgacctgttttgtctgccgctcggcgaatccttctccgcgctaaccgtctccgaacacgtcagttatgtcgtaacaaaccagtacctgatcaaaggtatctcctaccctgtctccaccaccgtcgtaggccagagcctcatcatcacccaaacggacagtcaaactaaatgcgaactaacgcgcaacatgcacaccacacacagcatcacagcggcgctcaacatttcactagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtcctagaagtaactgacgtcgtcgtggacgccaccgacagtcgtctcctcatgatgtccgtctacgcgctatcggccatcatcggcatctatctgctctaccgcatgctcaagacatgctga